(19) United States
(12) Patent Application Publication
(10) Pub. No.: US 2010/0284985 A1
(43) Pub. Date: Nov. 11, 2010

(54) Composition

(75) Inventors: Tina Mygind, Arhus N (DK); Arnar Sigmundsson, Arhus (DK); Jana Fischer, Brabrand (DK)

Correspondence Address: STEPTOE & JOHNSON LLP, 1330 CONNECTICUT AVENUE, N.W., WASHINGTON, DC, 20036, US

(73) Assignee: Danisco A/S

(21) Appl. No.: 12/676,112

(22) PCT Filed: Sep. 3, 2008
(86) PCT No.: PCT/IB2008/003067

§ 371 Date: Jun. 3, 2010

(30) Foreign Application Priority Data
Sep. 4, 2007 (GB) 0717182.0

(51) Int. Cl. A01N 65/00; A01N 65/08; C09D 5/16; A23L 3/34; A61K 38/16; A61P 31/04; A01P 1/00

(52) U.S. Cl. 424/94.1; 106/18.32; 424/725; 424/729; 424/732; 424/766; 426/335; 426/532; 514/2.9

(57) ABSTRACT

The present invention provides a composition comprising (a) an antimicrobial compound of the formula (I) wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄; (b) an antimicrobial material selected from lanthionine bacteriocins, tea [Camellia sinensis] extract, hop [Humulus lupulus L.] extract, grape skin extract, grape seed extract, Uva Ursi extract and combinations thereof.

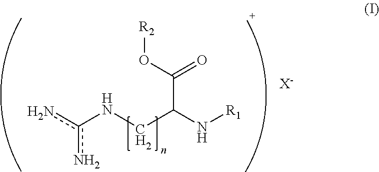

### BACKGROUND

Food safety and prevention of food spoilage is an ever present concern worldwide, particularly with the increasing trend for convenience foods such as ready to eat meals, soups, sauces or snacks. Spoilage of food is a major economic problem for the food manufacturer. Food manufacturers need to protect the health and safety of the public by delivering products that are safe to eat. Such food must have a guaranteed shelf life, either at chilled or ambient temperature storage. Consumers prefer good tasting food of high quality—this is difficult to achieve with chemical preservatives, harsh heating regimes and other processing measures. Food safety and protection is best achieved with a multiple preservation system using a combined approach of milder processing and natural preservatives. Foodborne micro-organisms are also less able to adapt and grow in food preserved with different preservative measures.

There is much concern about food safety and the growth of food pathogens such as Listeria monocytogenes. This particular pathogen can grow at low temperatures, which are often used as an additional preservative measure. Foodborne pathogens can sometimes adapt to different preservatives and storage conditions, thus a combination of preservative measures can be more successful than individual measures.

Bacteriocins are antimicrobial proteins or peptides that can be produced by certain bacteria, which can kill or inhibit the growth of closely related bacteria. The bacteriocins produced by lactic acid bacteria are of particular importance since they have great potential for the preservation of food and for the control of foodborne pathogens. (Wessels et al. 1998.)

The most well known bacteriocin is nisin, which is the only bacteriocin currently authorised as a food additive. Nisin is produced by fermentation of the dairy starter culture bacterium Lactococcus lactis subsp. lactis, and is sold as the commercial extract Nisaplin® Natural Antimicrobial (Danisco). Nisin has an unusually broad antimicrobial spectrum for a bacteriocin, being active against most Gram-positive bacteria (e.g. species of Bacillus, Clostridium, Listeria, lactic acid bacteria). It is not normally effective against Gram-negative bacteria, yeasts or moulds. Nisin is allowed as a food preservative worldwide but its levels of use and approved food applications are strictly regulated, varying from country to country.

Other bacteriocins have since been discovered with potential as food preservatives, e.g. pediocin, lacticin, sakacin, lactococcin, enterococin, plantaricin, leucocin. These are also active, although usually with a more narrow spectrum, against Gram-positive bacteria. Their food use is at present restricted to production of the bacteriocin in situ, i.e. by growth of the producer organism within the food.

LAE (also known as Mirenat-N, lauric arginate, N^{α}-Lauroyl-L-arginine ethyl ester monohydrochloride and lauramide arginine ethyl ester) is a cationic surfactant molecule chemically synthesised using the natural components; lauric acid, ethanol and L-arginine. The chemical structure is shown below

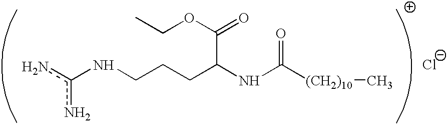

LAE has been shown to have a unique broad range of antimicrobial activity (1), and it has been shown to maintain this activity over a pH range between 3-7. LAE is heat stable during cooking processes and it has a shelf life of two years in powder form. The substance is water soluble, meaning that it is active in the water phase where most microorganisms reside. LAE is sold as a 10% solution in propylene glycol (propylene glycol is also GRAS).

LAE has limitations at least because it can precipitate in teas, grape and apple fruit drinks, it can lead to off flavour (bitter taste) and it is enzymatically degraded in fresh meat.

LAE exerts antimicrobial action on the cytoplasmic membrane, altering the membrane potential as determined by transmembrane ion flux (K⁺ and H⁺) measurements and causing structural membrane changes as determined by electron microscopy and fluorescence microscopy, but without complete disruption of cells (5).

LAE has been assessed by FDA and classified as GRAS (9), and USDA has approved it for use in meat and poultry (10) All studies on LAE and its hydrolysis products have shown that human consumption of LAE used as a preservative in foods and human exposure to LAE used as a preservative in cosmetics are safe.

There is an increasing need to develop economical, natural and effective preservative systems to meet the public demand for convenient, natural, safe, healthy, good quality products with guaranteed shelf life. Bacteriocins such as nisin can be used as preservatives in food to help meet this need. Nisin is a proven safe, natural preservative with GRAS status. There is also a continuing to desire to provide microbial protection utilising lower amounts of bacteriocins. Thus there is a need to provide new bacteriocins or new more effective combinations of bacteriocins.

In one aspect the present invention provides a composition comprising (a) an antimicrobial compound of the formula

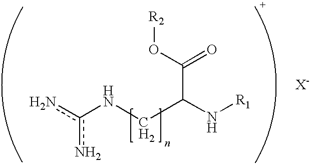

wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄⁻ (b) an antimicrobial material selected from lanthionine bacteriocins, macrolide antimicrobials, tea [Camellia sinensis] extract, hop [Humulus lupulus L.] extract, grape skin extract, grape seed extract, Uva Ursi extract and combinations thereof. Preferably the antimicrobial material is selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

In one aspect the present invention provides a process for preventing and/or inhibiting the growth of, and/or killing a micro-organism in a material, the process comprising the step of contacting the material with (a) an antimicrobial compound of the formula

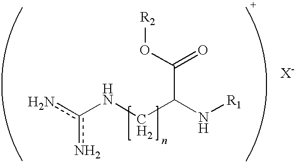

wherein R¹ is a fatty acid chain; R² is a linear or branched alkyl residue having from 1 to 12 carbon atoms; n is an integer from 0 to 10; X⁻ is selected from Br⁻, Cl⁻ and HSO₄⁻; (b) an antimicrobial material selected from lanthionine bacteriocins, macrolide antimicrobials, tea [Camellia sinensis] extract, hop [Humulus lupulus L.] extract, grape skin extract, grape seed extract, Uva Ursi extract and combinations thereof. Preferably the antimicrobial material selected from lanthionine bacteriocins, tea [Camellia sinensis] extract, hop [Humulus lupulus L.] extract, grape skin extract, grape seed extract, Uva Ursi extract and combinations thereof.

In one aspect the present invention provides use of (a) an antimicrobial compound of the formula

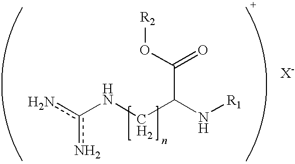

wherein R¹ is a fatty acid chain; R² is a linear or branched alkyl residue having from 1 to 12 carbon atoms; n is an integer from 0 to 10; X⁻ is selected from Br⁻, Cl⁻ and HSO₄⁻; and (b) an antimicrobial material selected from lanthionine bacteriocins, macrolide antimicrobials, tea [Camellia sinensis] extract, hop [Humulus lupulus L.] extract, grape skin extract, grape seed extract, Uva Ursi extract and combinations thereof; for preventing and/or inhibiting the growth of, and/or killing a micro-organism in a material. Preferably the antimicrobial material is selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

In one aspect the present invention provides a kit for preparing a composition of the invention, the kit comprising; (a) an antimicrobial compound of the formula;

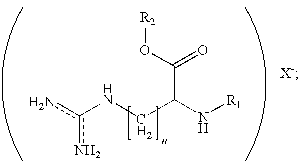

wherein R¹ is a fatty acid chain; R² is a linear or branched alkyl residue having from 1 to 12 carbon atoms; n is an integer from 0 to 10; X⁻ is selected from Br⁻, Cl⁻ and HSO₄⁻; (b) an antimicrobial material selected from lanthionine bacteriocins, macrolide antimicrobials, tea [Camellia sinensis] extract, hop [Humulus lupulus L.] extract, grape skin extract, grape seed extract, Uva Ursi extract and combinations thereof; in separate packages or containers; optionally with instructions for admixture and/or contacting and/or use. Preferably the antimicrobial material is selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

In one aspect the present invention provides a foodstuff comprising an antimicrobial additive composition comprising (a) an antimicrobial compound of the formula

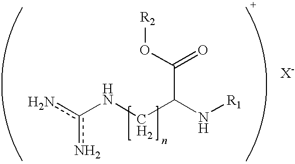

wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄⁻ (b) an antimicrobial material selected from lanthionine bacteriocins, macrolide antimicrobials, tea [Camellia sinensis] extract, hop [Humulus lupulus L.] extract, grape skin extract, grape seed extract, Uva Ursi extract and combinations thereof. Preferably the antimicrobial material is selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

In one aspect the present invention provides an antimicrobial protected material comprising (i) a material to be protected from microbial growth and (ii) an antimicrobial additive composition comprising (a) an antimicrobial compound of the formula

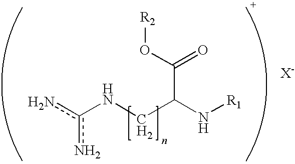

wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄⁻ (b) an antimicrobial material selected from lanthionine bacteriocins, macrolide antimicrobials, tea [Camellia sinensis] extract, hop [Humulus lupulus L.] extract, grape skin extract, grape seed extract, Uva Ursi extract and combinations thereof. Preferably the antimicrobial material is selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

Further aspects of the invention are defined herein and in the appended claims.

The present invention provides a synergistic combination of components for preventing and/or inhibiting the growth of, and/or killing a micro-organism in a material, such as foodstuff. This combination of components allows lower levels of the antimicrobial material to be used to provide effective action and prevent the development of tolerance to the antimicrobial material. This is particularly important in food applications where reduction of dosage and/or avoidance of development of tolerance is desired for commercial and regulatory reasons.

For ease of reference, these and further aspects of the present invention are now discussed under appropriate section headings. However, the teachings under each section are not necessarily limited to each particular section.

Preferred Aspects

Antimicrobial Compound

As discussed herein the antimicrobial compound of the formula;

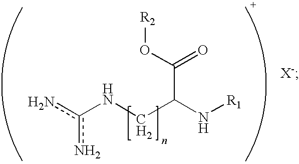

wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄⁻.

R¹ may be a linear or branched fatty acid chain. R¹ may be a branched fatty acid chain. R¹ is preferably a linear fatty acid chain.

The R¹ (linear or branched) fatty acid chain may be the chain of an unsaturated fatty acid or may be the alkyl chain of a saturated fatty. Preferably R¹ is a alkyl chain of a saturated fatty acid chain. In one preferred aspect R¹ is an alkyl chain of a linear saturated fatty acid chain.

In one preferred aspect the fatty acid chain/R¹ is the following group —C(═O)—(CH₂)p-CH₃ wherein p is from 2 to 20.

In one preferred aspect p is from 4 to 18, more preferably p is from 6 to 16, more preferably p is from 8 to 14, more preferably p is from 8 to 12, more preferably p is 10.

R² is a linear or branched alkyl residue having from 1 to 12 carbon atoms. In one preferred aspect R² is a linear or branched alkyl residue having from 1 to 8 carbon atoms, such as a linear or branched alkyl residue having from 1 to 4 carbon atoms or a linear alkyl residue having from 1, 2 or 3 carbon atoms. In one highly preferred aspect R² is an ethyl residue.

In a further preferred aspect R² is a alkyl residue having from 1 to 12 carbon atoms. In a yet further preferred aspect R² is a linear alkyl residue having from 1 to 8 carbon atoms, such as a linear alkyl residue having from 1 to 4 carbon atoms or a linear alkyl residue having from 1, 2 or 3 carbon atoms.

In the general formula

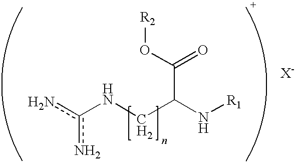

n is an integer from 0 to 10. Preferably n is an integer from 0 to 6, more preferably n is an integer from 1 to 4. In a highly preferred embodiment n is 3.

In the general formula

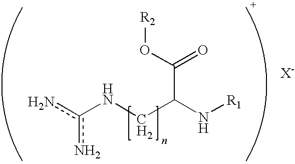

X⁻ is selected from Br⁻, I⁻, Cl⁻ and HSO₄⁻. Preferably X⁻ is Cl⁻. Thus in one preferred aspect the compound for use in the present invention is of the formula

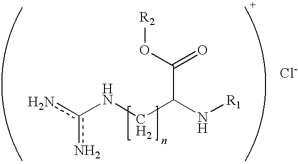

In a highly preferred aspect the antimicrobial compound is

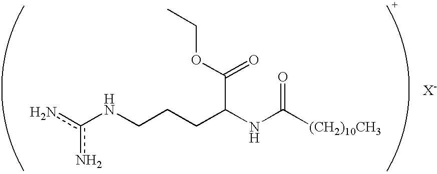

wherein X⁻ is selected from Br⁻, I⁻, Cl⁻ and HSO₄⁻.

In a highly preferred aspect the antimicrobial compound is

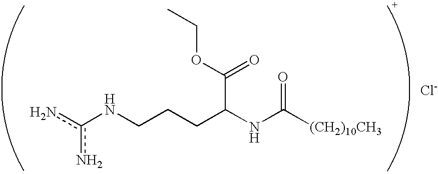

It will be appreciated that this compound is LAE as described herein

The antimicrobial compound may be present in any amount to provide the required microbicidal or microbiostatic effect. This effect may be typically be in the final material in which microbial growth is to be inhibited. Thus when the present invention provides an additive composition the antimicrobial compound may be present in an amount such that when the composition is added to the material to be ‘protected’ in the directed amounts, the antimicrobial compound is present in an amount in the material to be protected to provide the required microbicidal or microbiostatic effect

In one aspect the antimicrobial compound is present in an amount to provide a microbicidal or microbiostatic effect.

In one aspect the composition is an antimicrobial additive composition. In this and in other aspects preferably the composition comprises the antimicrobial compound in an amount of at least 0.5% based on the composition. The antimicrobial compound may be present in an amount of at least 1% based on the composition. The antimicrobial compound may be present in an amount of at least 2% based on the composition. The antimicrobial compound may be present in an amount of at least 5% based on the composition. The antimicrobial compound may be present in an amount of at least 10% based on the composition. Yet further the antimicrobial compound may be present in an amount of at least 15 wt. % based on the composition.

Antimicrobial Material

As discussed herein, the present invention utilises an antimicrobial material selected from lanthionine bacteriocins, macrolide antimicrobials, tea [Camellia sinensis] extract, hop [Humulus lupulus L.] extract, grape skin extract, grape seed extract, Uva Ursi extract and combinations thereof. Preferably the antimicrobial material selected from lanthionine bacteriocins, tea [Camellia sinensis] extract, hop [Humulus lupulus L.] extract, grape skin extract, grape seed extract, Uva Ursi extract and combinations thereof. Preferably the antimicrobial material selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

The antimicrobial material may be present in any amount to provide the required microbicidal or microbiostatic effect. This effect may be typically be in the final material in which microbial growth is to be inhibited. Thus when the present invention provides an additive composition the antimicrobial material may be present in an amount such that when the composition is added to the material to be ‘protected’ in the directed amounts, the antimicrobial material is present in an amount in the material to be protected to provide the required microbicidal or microbiostatic effect

In one aspect the antimicrobial material is present in an amount to provide a microbicidal or microbiostatic effect.

In one aspect the composition is an antimicrobial additive composition. In this and in other aspects preferably the composition comprises the antimicrobial material in an amount of at least 10% based on the composition. The antimicrobial material may be present in an amount of at least 20% based on the composition. The antimicrobial material may be present in an amount of at least 30% based on the composition. The antimicrobial material may be present in an amount of at least 40% based on the composition. The antimicrobial material may be present in an amount of at least 50% based on the composition. The antimicrobial material may be present in an amount of at least 60% based on the composition. The antimicrobial material may be present in an amount of at least 70% based on the composition. Yet further the antimicrobial material may be present in an amount of at least 80 wt. % based on the composition.

The amount of antimicrobial compound and the amount of antimicrobial material may depend on the application in which the system is to be utilised, the microorganism against which action is desired and/or the choice of antimicrobial material. Amounts and ratios of antimicrobial compound and antimicrobial material are given below based on the antimicrobial material used:
when the antimicrobial material is a lanthionine bacteriocin (preferably nisin), preferably the antimicrobial material is present in an amount of 30-70 wt % based on the total composition and the antimicrobial compound is present in an amount of 70-30 wt % based on the total composition
when the antimicrobial material is a lanthionine bacteriocin (preferably nisin), preferably the antimicrobial material is present in an amount of 30-70 wt % based on the total amount of antimicrobial compound and antimicrobial material, and the antimicrobial compound is present in an amount of 70-30 wt % based on the total amount of antimicrobial compound and antimicrobial material
when the antimicrobial material is a lanthionine bacteriocin (preferably nisin), preferably the antimicrobial material is present in an amount of 40-60 wt % based on the total composition and the antimicrobial compound is present in an amount of 60-40 wt % based on the total composition
when the antimicrobial material is a lanthionine bacteriocin (preferably nisin), preferably the antimicrobial material is present in an amount of 40-60 wt % based on the total amount of antimicrobial compound and antimicrobial material, and the antimicrobial compound is present in an amount of 60-40 wt % based on the total amount of antimicrobial compound and antimicrobial material
when the antimicrobial material is a lanthionine bacteriocin (preferably nisin), preferably the antimicrobial material is present in an amount of approximately 50 wt % based on the total composition and the antimicrobial compound is present in an amount of approximately 50 wt % based on the total composition
when the antimicrobial material is a lanthionine bacteriocin (preferably nisin), preferably the antimicrobial material is present in an amount of approximately 50 wt % based on the total amount of antimicrobial compound and antimicrobial material, and the antimicrobial compound is present in an amount of approximately 50 wt % based on the total amount of antimicrobial compound and antimicrobial material
when the antimicrobial material is a tea extract, preferably the antimicrobial material is present in an amount of 98-99.9 wt % based on the total composition and the antimicrobial compound is present in an amount of 0.1-2 wt % based on the total composition
when the antimicrobial material is a tea extract, preferably the antimicrobial material is present in an amount of 98-99.9 wt % based on the total amount of antimicrobial compound and antimicrobial material, and the antimicrobial compound is present in an amount of 0.1-2 wt % based on the total amount of antimicrobial compound and antimicrobial material
when the antimicrobial material is a tea extract, preferably the antimicrobial material is present in an amount of 98-99 wt % based on the total composition and the antimicrobial compound is present in an amount of 1-2 wt % based on the total composition
when the antimicrobial material is a tea extract, preferably the antimicrobial material is present in an amount of 98-99 wt % based on the total amount of antimicrobial compound and antimicrobial material, and the antimicrobial compound is present in an amount of 1-2 wt % based on the total amount of antimicrobial compound and antimicrobial material
when the antimicrobial material is a macrolide antimicrobial (preferably natamycin), preferably the antimicrobial material is present in an amount of 1-20 wt % based on the total composition and the antimicrobial compound is present in an amount of 99-80 wt % based on the total composition
when the antimicrobial material is a macrolide antimicrobial (preferably natamycin), preferably the antimicrobial material is present in an amount of 1-20 wt % based on the total amount of antimicrobial compound and antimicrobial material, and the antimicrobial compound is present in an amount of 99-80 wt % based on the total amount of antimicrobial compound and antimicrobial material
when the antimicrobial material is a macrolide antimicrobial (preferably natamycin), preferably the antimicrobial material is present in an amount of 5-15 wt % based on the total composition and the antimicrobial compound is present in an amount of 95-85 wt % based on the total composition
when the antimicrobial material is a macrolide antimicrobial (preferably natamycin), preferably the antimicrobial material is present in an amount of 5-15 wt % based on the total amount of antimicrobial compound and antimicrobial material, and the antimicrobial compound is present in an amount of 95-85 wt % based on the total amount of antimicrobial compound and antimicrobial material
when the antimicrobial material is a macrolide antimicrobial (preferably natamycin), preferably the antimicrobial material is present in an amount of 8-12 wt % based on the total composition and the antimicrobial compound is present in an amount of 92-88 wt % based on the total composition
when the antimicrobial material is a macrolide antimicrobial (preferably natamycin), preferably the antimicrobial material is present in an amount of 8-12 wt % based on the total amount of antimicrobial compound and antimicrobial material, and the antimicrobial compound is present in an amount of 92-88 wt % based on the total amount of antimicrobial compound and antimicrobial material
when the antimicrobial material is a macrolide antimicrobial (preferably natamycin), preferably the antimicrobial material is present in an amount of approximately 10 wt % based on the total composition and the antimicrobial compound is present in an amount of approximately 90 wt % based on the total composition
when the antimicrobial material is a macrolide antimicrobial (preferably natamycin), preferably the antimicrobial material is present in an amount of approximately 10 wt % based on the total amount of antimicrobial compound and antimicrobial material, and the antimicrobial compound is present in an amount of approximately 90 wt % based on the total amount of antimicrobial compound and antimicrobial material
when the antimicrobial material is a grape seed extract, preferably the antimicrobial material is present in an amount of 98-99.9 wt % based on the total composition and the antimicrobial compound is present in an amount of 0.1-2 wt % based on the total composition
when the antimicrobial material is a grape seed extract, preferably the antimicrobial material is present in an amount of 98-99.9 wt % based on the total amount of antimicrobial compound and antimicrobial material, and the antimicrobial compound is present in an amount of 0.1-2 wt % based on the total amount of antimicrobial compound and antimicrobial material
when the antimicrobial material is a grape seed extract, preferably the antimicrobial material is present in an amount of 98-99 wt % based on the total composition and the antimicrobial compound is present in an amount of 1-2 wt % based on the total composition
when the antimicrobial material is a grape seed extract, preferably the antimicrobial material is present in an amount of 98-99 wt % based on the total amount of antimicrobial compound and antimicrobial material, and the antimicrobial compound is present in an amount of 1-2 wt % based on the total amount of antimicrobial compound and antimicrobial material
when the antimicrobial material is a grape skin extract, preferably the antimicrobial material is present in an amount of 98-99.9 wt % based on the total composition and the antimicrobial compound is present in an amount of 0.1-2 wt % based on the total composition
when the antimicrobial material is a grape skin extract, preferably the antimicrobial material is present in an amount of 98-99.9 wt % based on the total amount of antimicrobial compound and antimicrobial material, and the antimicrobial compound is present in an amount of 0.1-2 wt % based on the total amount of antimicrobial compound and antimicrobial material
when the antimicrobial material is a grape skin extract, preferably the antimicrobial material is present in an amount of 98-99 wt % based on the total composition and the antimicrobial compound is present in an amount of 1-2 wt % based on the total composition
when the antimicrobial material is a grape skin extract, preferably the antimicrobial material is present in an amount of 98-99 wt % based on the total amount of antimicrobial compound and antimicrobial material, and the antimicrobial compound is present in an amount of 1-2 wt % based on the total amount of antimicrobial compound and antimicrobial material
when the antimicrobial material is a Uva ursi extract, preferably the antimicrobial material is present in an amount of 98-99.9 wt % based on the total composition and the antimicrobial compound is present in an amount of 0.1-2 wt % based on the total composition
when the antimicrobial material is a Uva ursi extract, preferably the antimicrobial material is present in an amount of 98-99.9 wt % based on the total amount of antimicrobial compound and antimicrobial material, and the antimicrobial compound is present in an amount of 0.1-2 wt % based on the total amount of antimicrobial compound and antimicrobial material
when the antimicrobial material is a Uva ursi extract, preferably the antimicrobial material is present in an amount of 98-99 wt % based on the total composition and the antimicrobial compound is present in an amount of 1-2 wt % based on the total composition
when the antimicrobial material is a Uva ursi extract, preferably the antimicrobial material is present in an amount of 98-99 wt % based on the total amount of antimicrobial compound and antimicrobial material, and the antimicrobial compound is present in an amount of 1-2 wt % based on the total amount of antimicrobial compound and antimicrobial material
when the antimicrobial material is a hops extract, preferably the antimicrobial material is present in an amount of 30-70 wt % based on the total composition and the antimicrobial compound is present in an amount of 70-30 wt % based on the total composition
when the antimicrobial material is a hops extract, preferably the antimicrobial material is present in an amount of 30-70 wt % based on the total amount of antimicrobial compound and antimicrobial material, and the antimicrobial compound is present in an amount of 70-30 wt % based on the total amount of antimicrobial compound and antimicrobial material
when the antimicrobial material is a hops extract, preferably the antimicrobial material is present in an amount of 40-60 wt % based on the total composition and the antimicrobial compound is present in an amount of 60-40 wt % based on the total composition
when the antimicrobial material is a hops extract, preferably the antimicrobial material is present in an amount of 40-60 wt % based on the total amount of antimicrobial compound and antimicrobial material, and the antimicrobial compound is present in an amount of 60-40 wt % based on the total amount of antimicrobial compound and antimicrobial material
when the antimicrobial material is a hops extract, preferably the antimicrobial material is present in an amount of approximately 50 wt % based on the total composition and the antimicrobial compound is present in an amount of approximately 50 wt % based on the total composition
when the antimicrobial material is a hops extract, preferably the antimicrobial material is present in an amount of approximately 50 wt % based on the total amount of antimicrobial compound and antimicrobial material, and the antimicrobial compound is present in an amount of approximately 50 wt % based on the total amount of antimicrobial compound and antimicrobial material

Lanthionine Bacteriocin

In one aspect the lanthionine bacteriocin is selected from nisin, sakacin and mixtures thereof. Preferably the lanthionine bacteriocin is nisin. Thus in one aspect the antimicrobial material is selected from nisin, macrolide antimicrobials, tea [Camellia sinensis] extract and combinations thereof. Preferably the antimicrobial material selected from nisin, tea [Camellia sinensis] extract and combinations thereof.

In one preferred aspect the antimicrobial material is at least nisin. In one preferred aspect the antimicrobial material consists of nisin.

Nisin is a lanthionine-containing bacteriocin (U.S. Pat. No. 5,691,301) derived from Lactococcus lactis subsp. lactis (formerly known as Streptococcus-lactis) (U.S. Pat. No. 5,573,801). In a preferred aspect of the present invention the bacteriocin used in the present invention is at least nisin.

As discussed in U.S. Pat. No. 5,573,801 nisin is a polypeptide bacteriocin produced by the lactic acid bacteria, Lactococcus lactis subsp. lactis (formerly known as Streptococcus lactis Group N).

Nisin is reportedly a collective name representing several closely related substances which have been designated nisin compounds A, B, C, D and E (De Vuyst, L. and Vandamme, E. J. 1994. Nisin, a lantibiotic produced by Lactococcus lactis subsp. lactis: properties, biosynthesis, fermentation and applications. In: Bacteriocins of lactic acid bacteria. Microbiology, Genetics and Applications. Eds.: De Vuyst and Vandamme. Blackie Academic and Professional, London). The structure and properties of nisin are also discussed in the article by E. Lipinska, entitled “Nisin and Its Applications”, The 25th Proceedings of the Easter School in Agriculture Science at the University of Nottingham, 1976, pp. 103-130 (1977), which article is hereby incorporated by reference. In 1969 the FAO/WHO Joint Expert Committee on Food Additives set specifications for the purity and identity of nisin (FAO/WHO Joint Expert Committee on Food Additives. 1969. Specifications for identity and purity of some antibiotics. 12^{th} Report. WHO Technical Report Series No. 430). This committee recognised nisin as a safe and legal preservative based on extensive toxicological testing. Nisin has the food additive number E234 and is classed as GRAS (Generally Recognised As Safe) (Food and Drug Administration. 1988. Nisin preparation: Affirmation of GRAS status as a direct human ingredient. Federal Regulations 53: 11247). The international activity unit (IU hereinafter) was defined as 0.001 mg of an international nisin reference preparation. Nisaplin® Natural Antimicrobial is the brand name for a nisin concentrate containing 1 million IU per g, which is commercially available from Danisco.

Nisin is an acknowledged and accepted food preservative with a long history of safe, effective food use. There have been several reviews of nisin, e.g. Hurst 1981; 1983; Delves-Broughton, 1990; De Vuyst and Vandamme, 1994; Thomas et al. 2000; Thomas & Delves-Broughton, 2001). Nisin was discovered over 50 years ago and the first commercial preparation, made in 1953, was Nisaplin®. Nisin has several characteristics that make it particularly suitable as a food preservative. It has undergone extensive toxicological testing to demonstrate its safety. It is heat-stable, acid-stable and effective against a broad spectrum of Gram-positive bacteria. It is not normally effective against Gram-negative bacteria, yeasts or moulds but activity against Gram-negative bacteria and yeasts has been reported in the presence of chelating agents (PCT/US 8902625. WO 89/12399). Nisin is an effective preservative in pasteurised and heat-treated foods (e.g. processed cheese, cheese, pasteurised milks, dairy desserts, cream, mascarpone and other dairy products, puddings such as semolina, tapioca etc., pasteurised liquid egg, pasteurised potato products, soy products, crumpets, pikelets, flapjacks, processed meat products, beverages, soups, sauces, ready to eat meals, canned foods, vegetable drinks) and low acid foods such as salad dressings, sauces, mayonnaise, beer, wine and other beverages.

Macrolide Antimicrobials

In one preferred aspect the antimicrobial material is at least a macrolide antimicrobial. In one preferred aspect the antimicrobial material consists of a macrolide antimicrobial.

In one preferred aspect the macrolide antimicrobial is at least natamycin. In one preferred aspect the macrolide antimicrobial is natamycin.

Natamycin is a polyene macrolide natural antifungal agent produced by fermentation of the bacterium Streptomyces natalensis. Natamycin (previously known as pimaricin) has an extremely effective and selective mode of action against a very broad spectrum of common food spoilage yeasts and moulds with most strains being inhibited by concentrations of 1-15 ppm of natamycin.

Natamycin is accepted as a food preservative and used world wide, particularly for surface treatment of cheese and dried fermented sausages. It has several advantages as a food preservative, including broad activity spectrum, efficacy at low concentrations, lack of resistance, and activity over a wide pH range. Neutral aqueous suspensions of natamycin are quite stable, but natamycin has poor stability in acid or alkaline conditions, in the presence of light, oxidants and heavy metals. For example, natamycin can be used in pasteurised fruit juice to prevent spoilage by heat-resistant moulds such as Byssochlamys. The acid pH of the juice, however, promotes degradation of natamycin during pasteurisation as well as during storage if the juice is not refrigerated. Natamycin is also degraded by high temperature heat processing, such as occurs during cooking of bakery items in an oven.

At extreme pH conditions, such as pH less than 4 and greater than 10, natamycin is rapidly inactivated with formation of various kinds of decomposition products. Acid hydrolysis of natamycin liberates the inactive aminosugar mycosamine. Further degradation reactions result in formation of dimers with a triene rather than a tetraene group. Heating at low pH may also result in decarboxylation of the aglycone. Alkaline hydrolysis results in saponification of the lactone. Both acid degradation products (aponatamycin, the aglycone dimer, and mycosamine), and alkaline or UV degradation products proved even safer than natamycin in toxicology tests, but are inactive biologically.

Tea Extract

As discussed herein the antimicrobial material may be or comprise tea [Camellia sinensis] extract. It will be understood by one skilled in the art that all references herein to tea extract mean an extract from a plant of the species Camellia sinensis.

It will be appreciated by one skilled in the art that by the term “extract” or “extracts” it is meant any constituent of the plant which may be isolated from the whole plant.

In a preferred aspect by the term tea “extract” or “extracts” of it is meant a leaf of the plant or a constituent which may be isolated from the leaf of whole plant.

In one preferred aspect the antimicrobial material is at least tea extract. In one preferred aspect the antimicrobial material consists of tea extract

In one preferred aspect the tea extract is a tea polyphenol. Preferably the tea extract is a catechin. In a highly preferred aspect the tea extract is a compound selected from

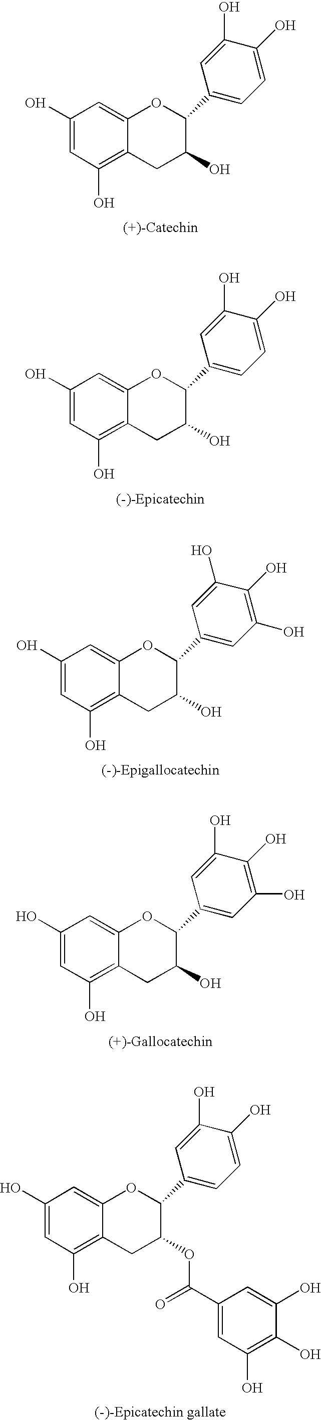

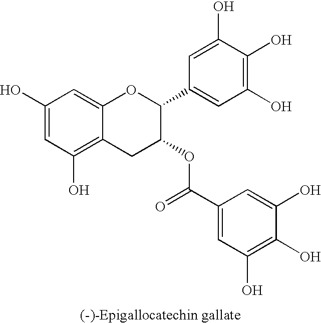

and mixtures thereof. It will be appreciated by one skilled in the art that the above compounds while ideally are isolated from a tea plant may be obtained by synthetic routes. Thus in one aspect the composition of the present invention or for use in the present invention comprises (a) an antimicrobial compound of the formula

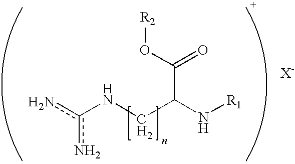

wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄⁻ (b) an antimicrobial material selected from
(i) lanthionine bacteriocins,
(ii) macrolide antimicrobials,
(iii) compounds selected from

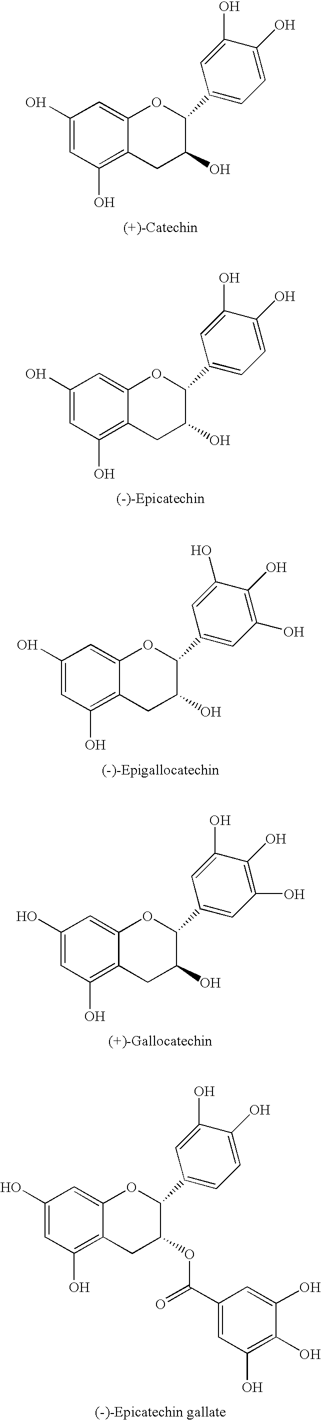

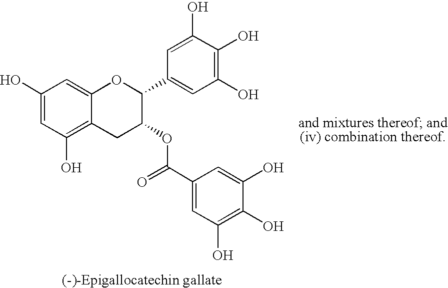

In one further aspect the composition of the present invention or for use in the present invention comprises (a) an antimicrobial compound of the formula

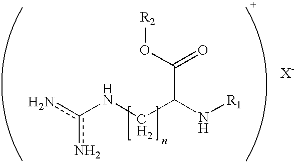

wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄⁻ (b) an antimicrobial material selected from
(i) lanthionine bacteriocins,
(ii) compounds selected from

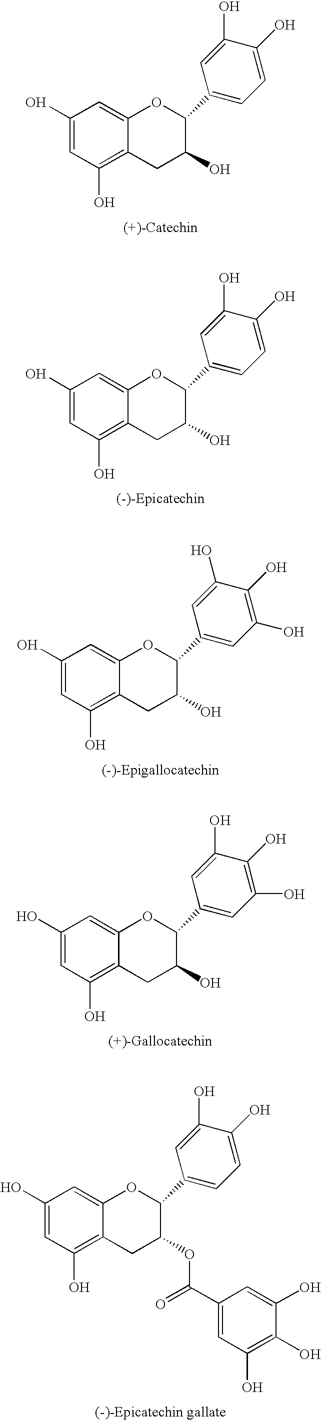

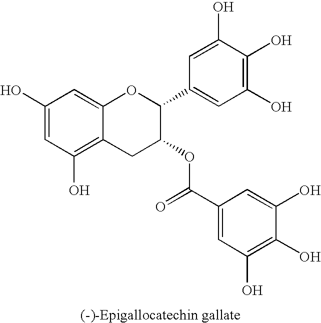

and mixtures thereof; and
(iii) combinations thereof.

Hop Extract

As discussed herein the antimicrobial material may be or comprise hop [Humulus lupulus L.] extract. It will be understood by one skilled in the art that all references herein to hop extract mean an extract from a plant of the species Humulus lupulus L.

It will be appreciated by one skilled in the art that by the term “extract” or “extracts” it is meant any constituent of the plant which may be isolated from the whole plant.

In one preferred aspect the antimicrobial material is at least hop extract. In one preferred aspect the antimicrobial material consists of hop extract

In one preferred aspect the hop extract is a hop alpha-acid (humulone), a hop beta-acid (lupulone), a derivative thereof or a mixture thereof. Derivatives of hop alpha-acids (humulones) and hop beta-acids (lupulones) include trans-humulone, cis-humulone, n-humulone, trans isohumulone, cis isohumulone, trans Rho isohumulone, trans tetrahydro isohumulone, and trans hexahydro isohumulone. Structures of and routes to these derivatives are shown below.

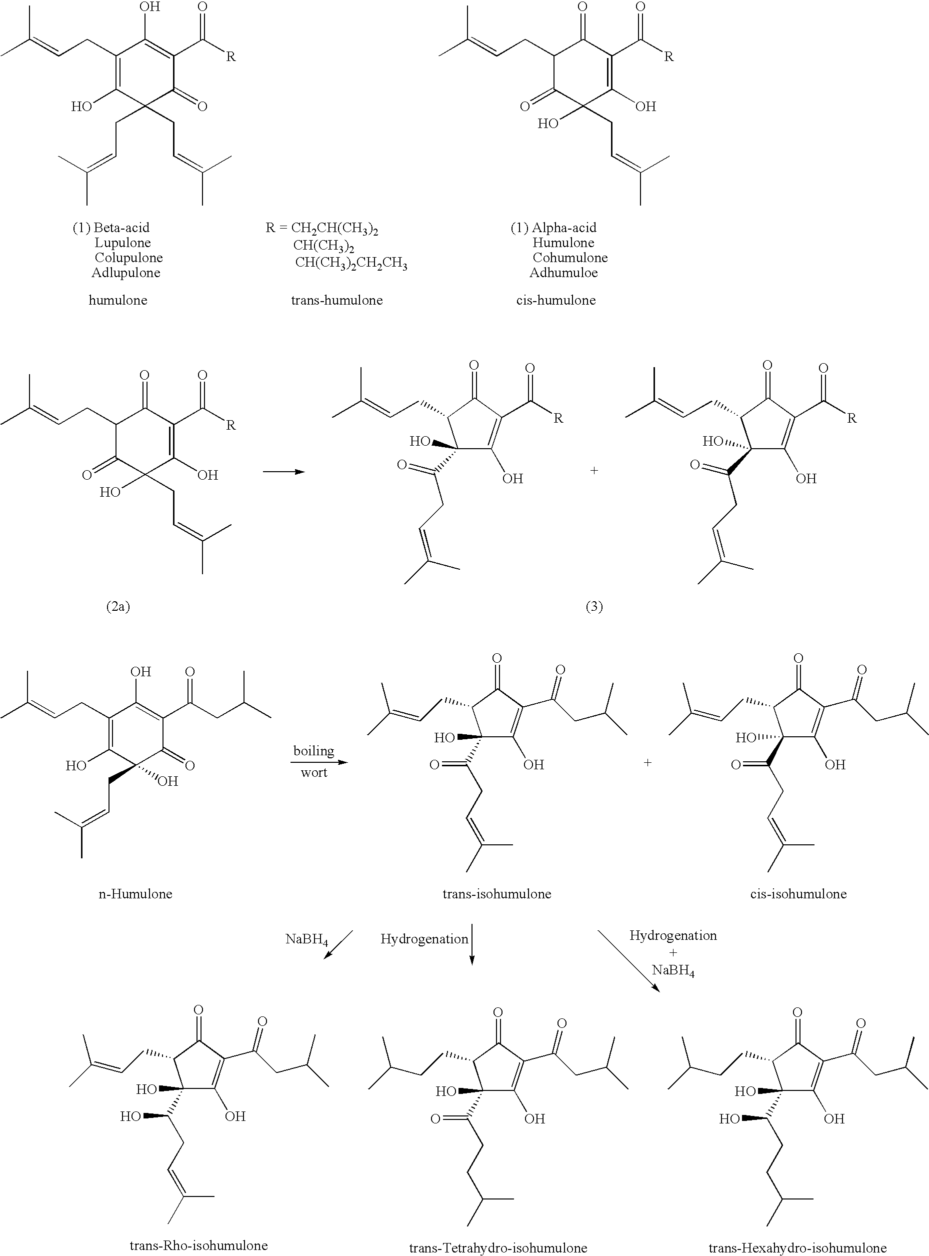

In one preferred aspect the hop extract is a hop alpha-acid (humulone), a hop beta-acid (lupulone), trans-humulone, cis-humulone, n-humulone, trans isohumulone, cis isohumulone, trans Rho isohumulone, trans tetrahydro isohumulone, trans hexahydro isohumulone, or a mixture thereof.

In the early 1900s, Brown and Clubb first described the antiseptic properties of hops. The most important component of Hop compounds, obtained from the female flower of the hop plant Humulus lupulus L. are so called hop bitter acids, which contribute to the characteristic bitterness and microbial stability. Subsequently, hop alpha-acids (humulones) and beta-acids (lupulones), constituents of the essential bitter resin of hop, were identified as strong antimicrobials mainly against Gram-positive bacteria.

Grape Skin Extract

As discussed herein the antimicrobial material may be or comprise grape skin extract. It will be understood by one skilled in the art that all references herein to grape mean the fruit of plants of the genus Vitis.

It will be appreciated by one skilled in the art that by the term “extract” or “extracts” it is meant any constituent of the grape skin which may be isolated from the whole grape skin.

In one preferred aspect the antimicrobial material is at least grape skin extract. In one preferred aspect the antimicrobial material consists of grape skin extract.

Grape Seed Extract

As discussed herein the antimicrobial material may be or comprise grape seed extract. It will be understood by one skilled in the art that all references herein to grape mean the fruit of plants of the genus Vitis.

It will be appreciated by one skilled in the art that by the term “extract” or “extracts” it is meant any constituent of the grape seed which may be isolated from the whole grape seed.

In one preferred aspect the antimicrobial material is at least grape seed extract. In one preferred aspect the antimicrobial material consists of grape seed extract.

Grape Extract

As will be appreciated by one skilled in the art, it has been shown by the present invention that significant parts of grape may be used in the present invention. Thus it will be appreciated that in a broad aspect, any reference in the present specification to grape seed extract or grape skin extract may be read as grape extract. By grape extract it is meant an extract of the fruit of plants of the genus Vitis.

It will be appreciated by one skilled in the art that by the term “extract” or “extracts” it is meant any constituent of the grape which may be isolated from the whole grape.

In one preferred aspect the antimicrobial material is at least grape extract. In one preferred aspect the antimicrobial material consists of grape extract.

Thus in broad aspects of the invention, there is provided

a composition comprising (a) an antimicrobial compound of the formula

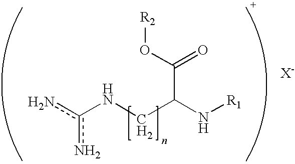

wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄⁻ (b) an antimicrobial material selected from lanthionine bacteriocins, macrolide antimicrobials, tea [Camellia sinensis] extract, hop [Humulus lupulus L.] extract, grape extract, Uva Ursi extract and combinations thereof. Preferably the antimicrobial material selected from lanthionine bacteriocins, tea [Camellia sinensis] extract and combinations thereof. Preferably the antimicrobial material is selected from lanthionine bacteriocins, macrolide antimicrobials, tea [Camellia sinensis] extract and combinations thereof.

a process for preventing and/or inhibiting the growth of, and/or killing a micro-organism in a material, the process comprising the step of contacting the material with (a) an antimicrobial compound of the formula

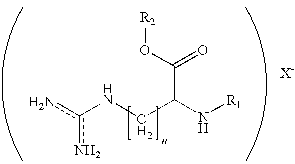

wherein R¹ is a fatty acid chain; R² is a linear or branched alkyl residue having from 1 to 12 carbon atoms; n is an integer from 0 to 10; X⁻ is selected from Br⁻, Cl⁻ and HSO₄⁻; (b) an antimicrobial material selected from lanthionine bacteriocins, macrolide antimicrobials, tea [Camellia sinensis] extract, hop [Humulus lupulus L.] extract, grape extract, Uva Ursi extract and combinations thereof. Preferably the antimicrobial material selected from lanthionine bacteriocins, tea [Camellia sinensis] extract, hop [Humulus lupulus L.] extract, grape extract, Uva Ursi extract and combinations thereof.

use of (a) an antimicrobial compound of the formula

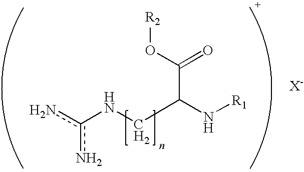

wherein R¹ is a fatty acid chain; R² is a linear or branched alkyl residue having from 1 to 12 carbon atoms; n is an integer from 0 to 10; X⁻ is selected from Br⁻, Cl⁻ and HSO₄⁻; and (b) an antimicrobial material selected from lanthionine bacteriocins, macrolide antimicrobials, tea [Camellia sinensis] extract, hop [Humulus lupulus L.] extract, grape extract, Uva Ursi extract and combinations thereof; for preventing and/or inhibiting the growth of, and/or killing a micro-organism in a material. Preferably the antimicrobial material is selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

a kit for preparing a composition of the invention, the kit comprising; (a) an antimicrobial compound of the formula;

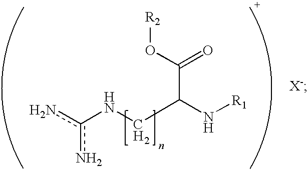

wherein R¹ is a fatty acid chain; R² is a linear or branched alkyl residue having from 1 to 12 carbon atoms; n is an integer from 0 to 10; X⁻ is selected from Br⁻, Cl⁻ and HSO₄⁻; (b) an antimicrobial material selected from lanthionine bacteriocins, macrolide antimicrobials, tea [Camellia sinensis] extract, hop [Humulus lupulus L.] extract, grape extract, Uva Ursi extract and combinations thereof; in separate packages or containers; optionally with instructions for admixture and/or contacting and/or use. Preferably the antimicrobial material is selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

a foodstuff comprising an antimicrobial additive composition comprising (a) an antimicrobial compound of the formula

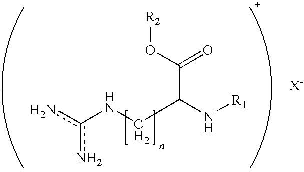

wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄⁻ (b) an antimicrobial material selected from lanthionine bacteriocins, macrolide antimicrobials, tea [Camellia sinensis] extract, hop [Humulus lupulus L.] extract, grape extract, Uva Ursi extract and combinations thereof. Preferably the antimicrobial material is selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

an antimicrobial protected material comprising (i) a material to be protected from microbial growth and (ii) an antimicrobial additive composition comprising (a) an antimicrobial compound of the formula

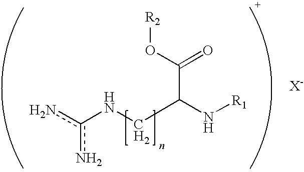

wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄⁻ (b) an antimicrobial material selected from lanthionine bacteriocins, macrolide antimicrobials, tea [Camellia sinensis] extract, hop [Humulus lupulus L.] extract, grape extract, Uva Ursi extract and combinations thereof. Preferably the antimicrobial material is selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

Uva Ursi Extract

As discussed herein the antimicrobial material may be or comprise Uva Ursi extract. It will be understood by one skilled in the art that all references herein to Uva Ursi extract mean an extract from a plant of the species Arctostaphylos uva-ursi.

It will be appreciated by one skilled in the art that plant of the species Arctostaphylos uva-ursi are of the genus Arctostaphylos. Other species of the genus Arctostaphylos may also provide the activity of the present invention. Thus in one broad aspect all references herein to Uva Ursi extract mean an extract from a plant of the genus Arctostaphylos.

It will be appreciated by one skilled in the art that by the term “extract” or “extracts” it is meant any constituent of the plant which may be isolated from the whole plant.

In a preferred aspect by the term Uva Ursi “extract” or “extracts” it is meant a leaf of the plant or a constituent which may be isolated from the leaf of whole plant.

In one preferred aspect the antimicrobial material is at least Uva Ursi extract. In one preferred aspect the antimicrobial material consists of Uva Ursi extract

In a highly preferred aspect the present invention provides a composition comprising

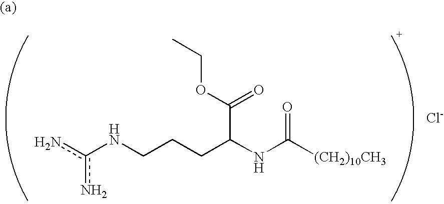

(b) nisin

In a highly preferred aspect the present invention provides a composition comprising

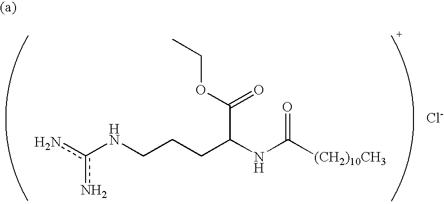

(b) natamycin

In a highly preferred aspect the present invention provides a composition comprising

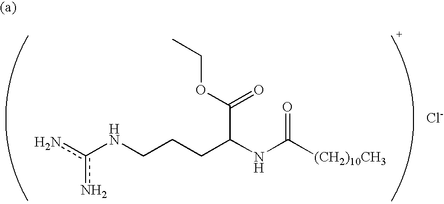

(b) a compound selected from

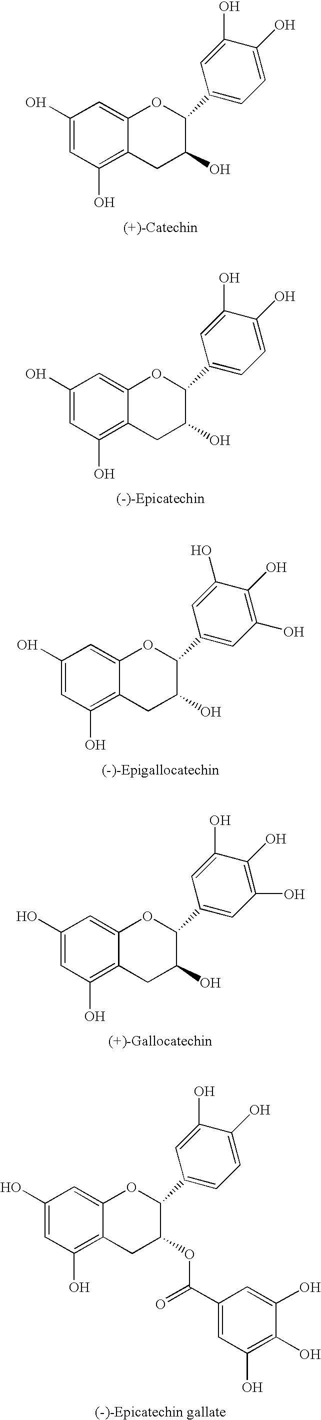

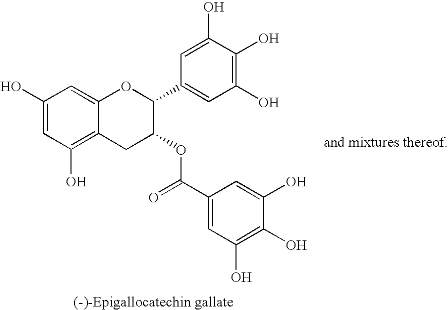

In a highly preferred aspect the present invention provides a composition comprising

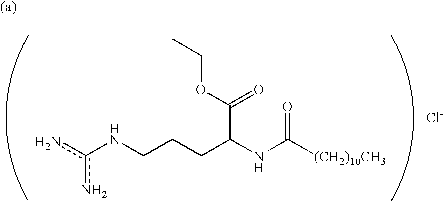

(b) hop [Humulus lupulus L.] extract.

In a highly preferred aspect the present invention provides a composition comprising

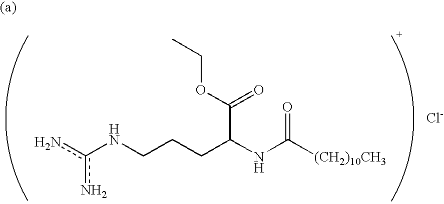

(b) grape skin extract.

In a highly preferred aspect the present invention provides a composition comprising

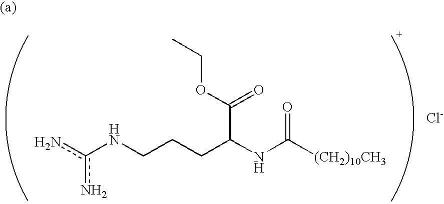

(b) grape seed extract.

In a highly preferred aspect the present invention provides a composition comprising

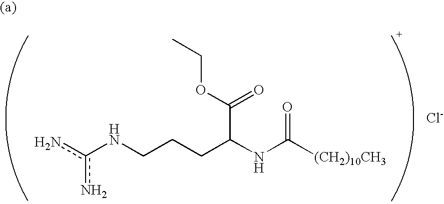

(b) Uva Ursi extract.

Micro-Organisms

In the context of the present invention the term “antimicrobial” is intended to mean that there is a bactericidal and/or a bacteriostatic and/or fungicidal and/or fungistatic effect and/or a virucidal effect, wherein

The term “bactericidal” is to be understood as capable of killing bacterial cells.

The term “bacteriostatic” is to be understood as capable of inhibiting bacterial growth, i.e. inhibiting growing bacterial cells.

The term “fungicidal” is to be understood as capable of killing fungal cells.

The term “fungistatic” is to be understood as capable of inhibiting fungal growth, i.e. inhibiting growing fungal cells.

The term “virucidal” is to be understood as capable of inactivating virus.

The term “microbial cells” denotes bacterial or fungal cells, and the term microorganism denotes a fungus (including yeasts) or a bacterium.

In the context of the present invention the term “inhibiting growth of microbial cells” is intended to mean that the cells are in the non-growing state, i.e., that they are not able to propagate.

As discussed herein the present invention may prevent and/or inhibit the growth of, and/or kill a micro-organism in a material. This may be slowing or arresting a micro-organism, such a bacteria, or by killing the micro-organism present on contact with the present composition.

In one aspect the antimicrobial compound and/or the antimicrobial material are present in an amount to provide a microbicidal or microbiostatic effect.

In one aspect the antimicrobial compound and the antimicrobial material are present in an amount to provide a microbicidal or microbiostatic effect.

In one aspect the antimicrobial compound and the antimicrobial material are present in an amount to provide a microbicidal or microbiostatic synergistic effect.

In one aspect the antimicrobial compound and the antimicrobial material are present in an amount to provide a microbicidal synergistic effect.

In a highly preferred aspect the microbicidal or microbiostatic effect is a bactericidal or bacteriostatic effect.

It is advantageous for the bactericidal or bacteriostatic effect to be in respect of Gram-positive bacteria and Gram-negative bacteria. Preferably the bactericidal or bacteriostatic effect is in respect of Gram-positive bacteria.

In a preferred aspect the bactericidal or bacteriostatic effect is in respect of an organism selected from species of Bacillus, species of Clostridium, species of Listeria, and species of Brochotrix.

In a preferred aspect the bactericidal or bacteriostatic effect is in respect of an organism selected from Gram-positive bacteria associated with food spoilage or foodborne disease including Bacillus species, Bacillus subtilis, Bacillus cereus, Listeria species, Listeria monocytogenes, lactic acid bacteria, lactic acid spoilage bacteria, Lactobacillus species, Staphylococcus aureus, Clostridium species, C. sporogenes, C. tyrobutyricum.

In a preferred aspect the bactericidal or bacteriostatic effect of the invention in combination with a chelating agent is in respect of an organism selected from other micro-organisms associated with food spoilage or foodborne disease, including yeasts, moulds and Gram-negative bacteria including Escherichia coli, Salmonella species, and Pseudomonas species.

In a preferred aspect the bactericidal or bacteriostatic effect is in respect of an organism selected from Bacillus cereus 204, B. cereus Campden, B. cereus NCTC2599, B. subtilis Campden, Clostridium sporogenes strain Campden, Clostridium sporogenes strain 1.221, Clostridium sporogenes NCIMB1793, Listeria monocytogenes 272, L. monocytogenes NCTC12426, L. monocytogenes S23, Lactobacillus sake 272, Escherichia coli S15, E. coli CRA109, Salmonella Typhimurium S29, Pseudomonas fluorescens 3756,

In a preferred aspect the bactericidal or bacteriostatic effect is in respect of Staphylococcus aureus, Listeria monocytogenes or combinations thereof.

In a preferred aspect the bactericidal or bacteriostatic effect is in respect of Staphylococcus aureus.

In a preferred aspect the bactericidal or bacteriostatic effect is in respect of Listeria monocytogenes.

Foodstuff

The composition, process and use of the present invention may prevent and/or inhibit the growth of, and/or kill a micro-organism in any material. However, in view of the problems associated with spoilage and contamination of foodstuffs and in view of the particular effectiveness of the present invention in foodstuffs, preferably the composition is a foodstuff or may be added to a foodstuff. It will be appreciated by one skilled in the art that when the present composition is a foodstuff the essential components of (a) an antimicrobial compound and (b) an antimicrobial material may already be present in the foodstuff. They may have been provided by one or more means. For example they may have been added in the form of a composition containing the antimicrobial compound and the antimicrobial material. The two components may have been added to the foodstuff sequentially. In one further aspect one or more of the components may have be formed in situ in the foodstuff. For example the antimicrobial material (such as nisin) may be formed in situ in the foodstuff by fermentation of the dairy starter culture bacterium Lactococcus lactis subsp. lactis.

The present invention may further encompass the use of an antimicrobial composition as defined herein in food and/or feed enzyme compositions, and may encompass food and/or feed compositions comprising an antimicrobial composition as defined herein. Such compositions may contain one or more further food ingredient or additives. By formulation of the antimicrobial composition of the invention within a food and/or feed composition, the composition can be stabilised to allow for prolonged storage (under suitable conditions) prior to use in food and/or feed production. In addition the antimicrobial composition of the present invention provides antimicrobials in a suitable form for safe use for the application in the preparation of foodstuffs and/or feedstuffs, or ingredients for use in food and/or feed preparation. Such compositions may be in either liquid, semi-liquid, crystalline, salts or solid/granular form.

In one aspect the composition of the present invention is an antimicrobial additive composition suitable for addition to a foodstuff.

In one aspect the present invention provides a foodstuff comprising an antimicrobial additive composition comprising (a) an antimicrobial compound of the formula

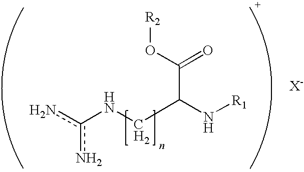

wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄⁻ (b) an antimicrobial material selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

Many foodstuffs may be protected by the present invention. Typical foodstuffs are raw meat, cooked meat, raw poultry products, cooked poultry products, raw seafood products, cooked seafood products, ready to eat meals, pasta sauces, pasteurised soups, mayonnaise, salad dressings, oil-in-water emulsions, margarines, low fat spreads, water-in-oil emulsions, dairy products, cheese spreads, processed cheese, dairy desserts, flavoured milks, cream, fermented milk products, cheese, butter, condensed milk products, ice cream mixes, soya products, pasteurised liquid egg, bakery products, confectionery products, fruit products, and foods with fat-based or water-containing fillings.

The term “foodstuff” as used herein means a substance which is suitable for human and/or animal consumption.

Suitably, the term “foodstuff” as used herein may mean a foodstuff in a form which is ready for consumption. Alternatively or in addition, however, the term foodstuff as used herein may mean one or more food materials which are used in the preparation of a foodstuff. By way of example only, the term foodstuff encompasses both baked goods produced from dough as well as the dough used in the preparation of said baked goods.

In a preferred aspect the present invention provides a foodstuff as defined above wherein the foodstuff is selected from one or more of the following: eggs, egg-based products, including but not limited to mayonnaise, salad dressings, sauces, ice creams, egg powder, modified egg yolk and products made therefrom; baked goods, including breads, cakes, sweet dough products, laminated doughs, liquid batters, muffins, doughnuts, biscuits, crackers and cookies; confectionery, including chocolate, candies, caramels, halawa, gums, including sugar free and sugar sweetened gums, bubble gum, soft bubble gum, chewing gum and puddings; frozen products including sorbets, preferably frozen dairy products, including ice cream and ice milk; dairy products, including cheese, butter, milk, coffee cream, whipped cream, custard cream, milk drinks and yoghurts; mousses, whipped vegetable creams, meat products, including processed meat products; edible oils and fats, aerated and non-aerated whipped products, oil-in-water emulsions, water-in-oil emulsions, margarine, shortening and spreads including low fat and very low fat spreads; dressings, mayonnaise, dips, cream based sauces, cream based soups, beverages, spice emulsions and sauces.

Suitably the foodstuff in accordance with the present invention may be a “fine foods”, including cakes, pastry, confectionery, chocolates, fudge and the like.

In one aspect the foodstuff in accordance with the present invention may be a dough product or a baked product, such as a bread, a fried product, a snack, cakes, pies, brownies, cookies, noodles, snack items such as crackers, graham crackers, pretzels, and potato chips, and pasta.

In a further aspect, the foodstuff in accordance with the present invention may be a plant derived food product such as flours, pre-mixes, oils, fats, cocoa butter, coffee whitener, salad dressings, margarine, spreads, peanut butter, shortenings, ice cream, cooking oils.

In another aspect, the foodstuff in accordance with the present invention may be a dairy product, including butter, milk, cream, cheese such as natural, processed, and imitation cheeses in a variety of forms (including shredded, block, slices or grated), cream cheese, ice cream, frozen desserts, yoghurt, yoghurt drinks, butter fat, anhydrous milk fat, other dairy products. The enzyme according to the present invention may improve fat stability in dairy products.

In another aspect, the foodstuff in accordance with the present invention may be a food product containing animal derived ingredients, such as fish, seafood, processed meat products, sausages, ham, cooking oils, shortenings.

In a further aspect, the foodstuff in accordance with the present invention may be a beverage, a fruit, mixed fruit, a vegetable, beer or wine.

In another aspect, the foodstuff in accordance with the present invention may be an animal feed. Suitably, the animal feed may be a poultry feed.

In one aspect preferably the foodstuff is selected from one or more of the following: eggs, egg-based products, including mayonnaise, salad dressings, sauces, ice cream, egg powder, modified egg yolk and products made therefrom.

Preferably the foodstuff according to the present invention is a water containing foodstuff. Suitably the foodstuff may be comprised of 10-99% water, suitably 14-99%, suitably of 18-99% water, suitably of 20-99%, suitably of 40-99%, suitably of 50-99%, suitably of 70-99%, suitably of 75-99%.

The antimicrobial composition can be applied to the foodstuff by dipping, or surface coating the foodstuff either by spraying the composition on the surface of the food or by applying the composition to castings or coatings or eatable films.

In a further aspect, the composition can be mixed into the foodstuff.

The present invention may be used to protect any material against microbial growth or proliferation—the present invention is not limited to use in foodstuffs. Thus in a further aspect the present invention provides an antimicrobial protected material comprising (i) a material to be protected from microbial growth and (ii) an antimicrobial additive composition comprising (a) an antimicrobial compound of the formula

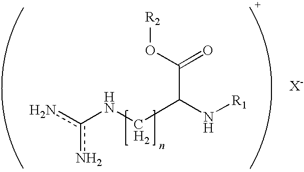

wherein R¹ is a fatty acid chain; R² is a linear or branched alkyl residue having from 1 to 12 carbon atoms; n is an integer from 0 to 10; X⁻ is selected from Br⁻, I⁻, Cl⁻ and HSO₄⁻ (b) an antimicrobial material selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

The antimicrobial protected material may be selected from any suitable material or surface. The antimicrobial protected material may be selected from a paint, an adhesive, an aqueous material and water.

The antimicrobial protected material may a hard surface. The term “hard surface” as used herein relates to any surface which is essentially non-permeable for microorganisms. Examples of hard surfaces are surfaces made from metal, e.g., stainless steel, plastics, rubber, board, glass, wood, paper, textile, concrete, rock, marble, gypsum and ceramic materials which optionally may be coated, e.g., with paint, enamel and the like. The hard surface can also be a process equipment, e.g., a cooling tower, an osmotic membrane, a water treatment plant, a dairy, a food processing plant, a chemical or pharmaceutical process plant.

The foodstuff or antimicrobial protected material may comprise the antimicrobial compound in an amount of no greater than 2000 ppm based on the composition. For example the foodstuff or antimicrobial protected material may comprise
the antimicrobial compound in an amount of no greater than 1000 ppm based on the composition, or
the antimicrobial compound in an amount of no greater than 500 ppm based on the composition, or
the antimicrobial compound in an amount of no greater than 200 ppm based on the composition, or
the antimicrobial compound in an amount of no greater than 100 ppm based on the composition.
tea extract in an amount of no greater than 20000 ppm based on the composition.
lanthionine bacteriocin in an amount of no greater than 500 ppm based on the composition.
nisin in an amount of no greater than 500 ppm based on the composition.
hop [Humulus lupulus L.] extract in an amount of no greater than 1000 ppm based on the composition.
hop [Humulus lupulus L.] extract in an amount of no greater than 500 ppm based on the composition.
hop [Humulus lupulus L.] extract in an amount of no greater than 50 ppm based on the composition.
grape skin extract in an amount of no greater than 5000 ppm based on the composition.
grape skin extract in an amount of no greater than 2500 ppm based on the composition.
grape seed extract in an amount of no greater than ????ppm based on the composition.
grape seed extract in an amount of no greater than 2500 ppm based on the composition.
Uva Ursi [Arctostaphylos uva-ursi] extract in an amount of no greater than 5000 ppm based on the composition.
Uva Ursi [Arctostaphylos uva-ursi] extract in an amount of no greater than 2500 ppm based on the composition.

In particular, in the foodstuff or antimicrobial protected material the composition may comprise
the compound

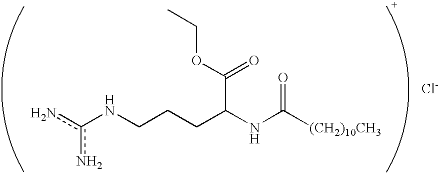

in an amount of no greater than 200 ppm based on the composition, and/or
the antimicrobial material in an amount of no greater than 20000 ppm based on the composition
the antimicrobial material in an amount of no greater than 10000 ppm based on the composition
the antimicrobial material in an amount of no greater than 5000 ppm based on the composition
the antimicrobial material in an amount of no greater than 2000 ppm based on the composition
the antimicrobial material in an amount of no greater than 500 ppm based on the composition
the antimicrobial material in an amount of no greater than 100 ppm based on the composition
the tea extract in an amount of no greater than 20000 ppm based on the composition
nisin in an amount of no greater than 500 ppm based on the composition.
macrolide antimicrobial in an amount of no greater than 100 ppm based on the composition.

Additional Components

The composition of the present invention or the composition for use in the present invention may contain one or more additional components. However, in some aspects the protectant composition of the present invention (suitable for addition to a foodstuff) contains no additional components or contains no additional components that materially affect the properties of the composition.

In one preferred aspect the composition further comprises an emulsifier. Preferably the emulsifier is selected from polyoxy-ethylene sorbitan esters (E432-E436) otherwise known as polysorbates (e.g. Tween 80, Tween 20), monoglycerides, diglycerides, acetic acid esters of mono-diglycerides, tartaric acid esters of mono-diglycerides and citric acid esters of mono-diglycerides.

In one preferred aspect the composition further comprises a chelator. Preferably the chelator is selected from EDTA, citric acid, monophosphates, diphosphates, triphosphates and polyphosphates.

Further suitable chelator are taught in U.S. Pat. No. 5,573,801 and include carboxylic acids, polycarboxylic acids, amino acids and phosphates. In particular, the following compounds and their salts may be useful:

Acetic acid, Adenine, Adipic acid, ADP, Alanine, B-Alanine, Albumin, Arginine, Ascorbic acid, Asparagine, Aspartic acid, ATP, Benzoic acid, n-Butyric acid, Casein, Citraconic acid, Citric acid, Cysteine, Dehydracetic acid, Desferri-ferrichrysin, Desferri-ferrichrome, Desferri-ferrioxamin E, 3,4-Dihydroxybenzoic acid, Diethylenetriaminepentaacetic acid (DTPA), Dimethylglyoxime, O,O-Dimethylpurpurogallin, EDTA, Formic acid, Fumaric acid, Globulin, Gluconic acid, Glutamic acid, Glutaric acid, Glycine, Glycolic acid, Glycylglycine, Glycylsarcosine, Guanosine, Histamine, Histidine, 3-Hydroxyflavone, Inosine, Inosine triphosphate, Iron-free ferrichrome, Isovaleric acid, Itaconic acid, Kojic acid, Lactic acid, Leucine, Lysine, Maleic acid, Malic acid, Methionine, Methylsalicylate, Nitrilotriacetic acid (NTA), Ornithine, Orthophosphate, Oxalic acid, Oxystearin, B-Phenylalanine, Phosphoric acid, Phytate, Pimelic acid, Pivalic acid, Polyphosphate, Proline, Propionic acid, Purine, Pyrophosphate, Pyruvic acid, Riboflavin, Salicylaldehyde, Salicyclic acid, Sarcosine, Serine, Sorbitol, Succinic acid, Tartaric acid, Tetrametaphosphate, Thiosulfate, Threonine, Trimetaphosphate, Triphosphate, Tryptophan, Uridine diphosphate, Uridine triphosphate, n-Valeric acid, Valine, and Xanthosine

Many of the above sequestering agents are useful in food processing in their salt forms, which are commonly alkali metal or alkaline earth salts such as sodium, potassium or calcium or quaternary ammonium salts. Sequestering compounds with multiple valencies may be beneficially utilised to adjust pH or selectively introduce or abstract metal ions e.g. in a food system coating. Additional information chelators is disclosed in T. E. Furia (Ed.), CRC Handbook of Food Additives, 2nd Ed., pp. 271-294 (1972, Chemical Rubber Co.), and M. S. Peterson and A. M. Johnson (Eds.), Encyclopaedia of Food Science, pp. 694-699 (1978, AVI Publishing Company, Inc.) which articles are both hereby incorporated by reference.

The terms “chelator” is defined as organic or inorganic compounds capable of forming co-ordination complexes with metals. Also, as the term “chelator” is used herein, it includes molecular encapsulating compounds such as cyclodextrin. The chelator may be inorganic or organic, but preferably is organic.

Preferred chelator are non-toxic to mammals and include aminopolycarboxylic acids and their salts such as ethylenediaminetetraacetic acid (EDTA) or its salts (particularly its di- and tri-sodium salts), and hydrocarboxylic acids and their salts such as citric acid. However, non-citric acid and non-citrate hydrocarboxylic acid chelators are also believed useful in the present invention such as acetic acid, formic acid, lactic acid, tartaric acid and their salts.

As noted above, the term “chelator” is defined and used herein as a synonym for sequestering agent and is also defined as including molecular encapsulating compounds such as cyclodextrin. Cyclodextrins are cyclic carbohydrate molecules having six, seven, or eight glucose monomers arranged in a donut shaped ring, which are denoted alpha, beta or gamma cyclodextrin, respectively. As used herein, cyclodextrin refers to both unmodified and modified cyclodextrin monomers and polymers. Cyclodextrin molecular encapsulators are commercially available from American Maize-Products of Hammond, Ind. Cyclodextrin are further described in Chapter 11 entitled, “Industrial Applications of Cyclodextrin”, by J. Szejtli, page 331-390 of Inclusion Compounds, Vol. III (Academic Press, 1984) which chapter is hereby incorporated by reference.

Preferably the chelator enhances the antimicrobial activity and/or antimicrobial spectrum of the bacteriocin. More preferably the chelator enhances the antimicrobial activity and/or antimicrobial spectrum of the bacteriocin in respect of Gram-negative bacteria and other micro-organisms.

In one preferred aspect the composition further comprises a lytic enzyme. Preferably the lytic enzyme is a lysozyme.

Process

As discussed herein in one aspect the present invention provides a process for preventing and/or inhibiting the growth of, and/or killing a micro-organism in a material, the process comprising the step of contacting the material with

(a) an antimicrobial compound of the formula

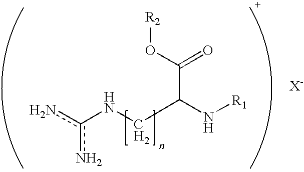

wherein R¹ is a fatty acid chain

R² is a linear or branched alkyl residue having from 1 to 12 carbon atoms

n is an integer from 0 to 10

X⁻ is selected from Br⁻, Cl⁻ and HSO₄⁻

(b) an antimicrobial material selected from lanthionine bacteriocins, macrolide antimicrobials, tea [Camellia sinensis] extract and combinations thereof. Preferably the antimicrobial material selected from lanthionine bacteriocins, tea [Camellia sinensis] extract and combinations thereof.

As discussed herein in one aspect the present invention provides use of

(a) an antimicrobial compound of the formula

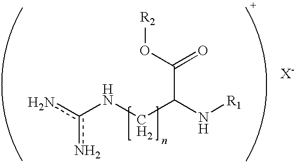

wherein R¹ is a fatty acid chain

R² is a linear or branched alkyl residue having from 1 to 12 carbon atoms

n is an integer from 0 to 10

X⁻ is selected from Br⁻, Cl⁻ and HSO₄⁻; and

(b) an antimicrobial material selected from lanthionine bacteriocins, macrolide antimicrobials, tea [Camellia sinensis] extract and combinations thereof;

for preventing and/or inhibiting the growth of, and/or killing a micro-organism in a material. Preferably the antimicrobial material selected from lanthionine bacteriocins, tea [Camellia sinensis] extract and combinations thereof.

In one aspect the antimicrobial compound and the antimicrobial material are added to the material together.

In one aspect the antimicrobial compound and the antimicrobial material are added to the material sequentially.

Thus the present invention provides in one aspect a preservative/protectant composition which may be added to a range of materials such as food systems and in another aspect a combination of two separate products which may added sequentially to materials such as food products.

In one aspect the extract is added to the material.

In one aspect the bacteriocin is added to the material.

In one aspect the antimicrobial material is formed in situ in the material. Preferably when the bacteriocin is nisin, the bacteriocin may be formed in situ in the foodstuff by fermentation of the dairy starter culture bacterium Lactococcus lactis subsp. lactis.

Further broad aspects of the present invention are defined below:

It has been found during our work that synergy may be observed in combinations of tea extract and an antimicrobial material selected from lanthionine bacteriocins and macrolide antimicrobials.

In a further aspect the present invention provides a composition comprising

(a) antimicrobial material selected from lanthionine bacteriocins and macrolide antimicrobials, preferably at least a lanthionine bacteriocin

(b) tea [Camellia sinensis] extract. The preferred aspects described herein in respect of antimicrobial material selected from lanthionine bacteriocins and macrolide antimicrobials and described herein in respect of tea extract, apply equally to this aspect of the invention.

In a further aspect the present invention provides a process for preventing and/or inhibiting the growth of, and/or killing a micro-organism in a material, the process comprising the step of contacting the material with (a) antimicrobial material selected from lanthionine bacteriocins and macrolide antimicrobials, preferably at least a lanthionine bacteriocin,

(b) tea [Camellia sinensis] extract. The preferred aspects described herein in respect of antimicrobial material selected from lanthionine bacteriocins and macrolide antimicrobials described herein in respect of tea extract, apply equally to this aspect of the invention.

In a further aspect the present invention provides use of (a) antimicrobial material selected from lanthionine bacteriocins and macrolide antimicrobials, preferably at least a lanthionine bacteriocin, and

(b) tea [Camellia sinensis] extract;

for preventing and/or inhibiting the growth of, and/or killing a micro-organism in a material.

The preferred aspects described herein in respect of antimicrobial material selected from lanthionine bacteriocins and macrolide antimicrobials and described herein in respect of tea extract, apply equally to this aspect of the invention.

In a further aspect the present invention provides a kit for preparing a composition (a) antimicrobial material selected from lanthionine bacteriocins and macrolide antimicrobials and (b) tea [Camellia sinensis] extract,

the kit comprising

(a) antimicrobial material selected from lanthionine bacteriocins and macrolide antimicrobials, preferably at least a lanthionine bacteriocin, and

(b) tea [Camellia sinensis] extract;

in separate packages or containers; optionally with instructions for admixture and/or contacting and/or use. The preferred aspects described herein in respect of antimicrobial material selected from lanthionine bacteriocins and macrolide antimicrobials and described herein in respect of tea extract, apply equally to this aspect of the invention.

In a further aspect the present invention provides a foodstuff comprising an antimicrobial additive composition comprising (a) antimicrobial material selected from lanthionine bacteriocins and macrolide antimicrobials, preferably at least a lanthionine bacteriocin, and (b) tea [Camellia sinensis] extract. The preferred aspects described herein in respect of antimicrobial material selected from lanthionine bacteriocins and macrolide antimicrobials and described herein in respect of tea extract, apply equally to this aspect of the invention.

In a further aspect the present invention provides an antimicrobial protected material comprising (i) a material to be protected from microbial growth and (ii) an antimicrobial additive composition comprising (a) antimicrobial material selected from lanthionine bacteriocins and macrolide antimicrobials, preferably at least a lanthionine bacteriocin, and (b) tea [Camellia sinensis] extract. The preferred aspects described herein in respect of antimicrobial material selected from lanthionine bacteriocins and macrolide antimicrobials and described herein in respect of tea extract, apply equally to this aspect of the invention.

The present invention will now be described in further detail by way of example only with reference to the accompanying figures in which:

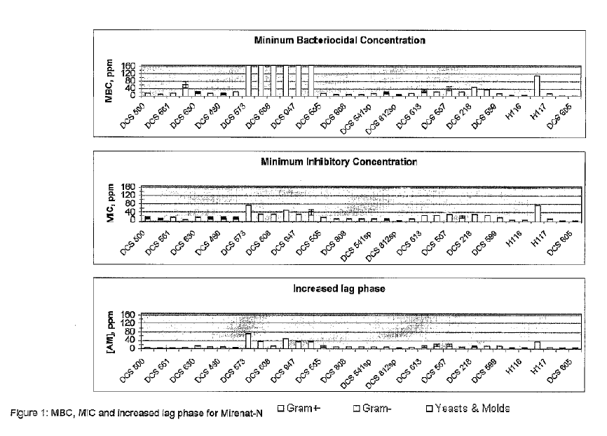

FIG. 1 is a graph;

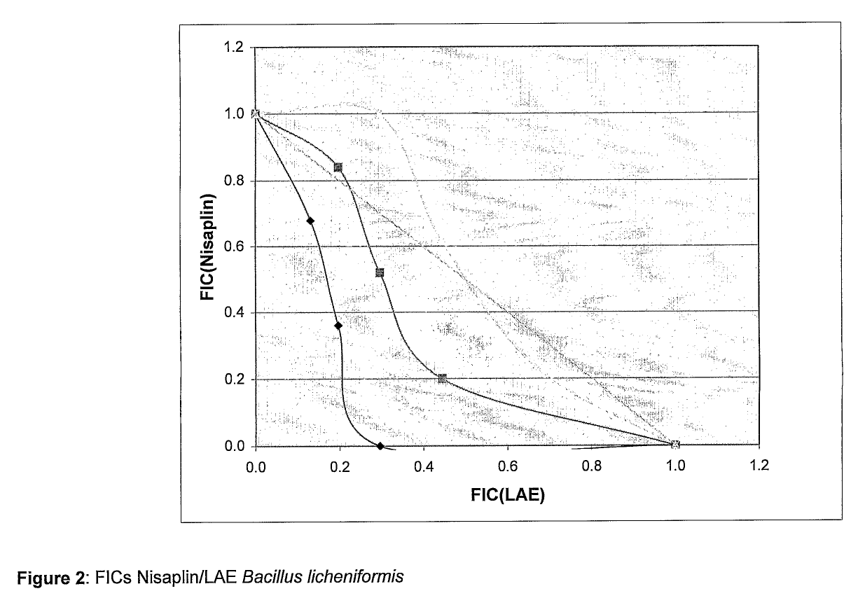

FIG. 2 is a graph;

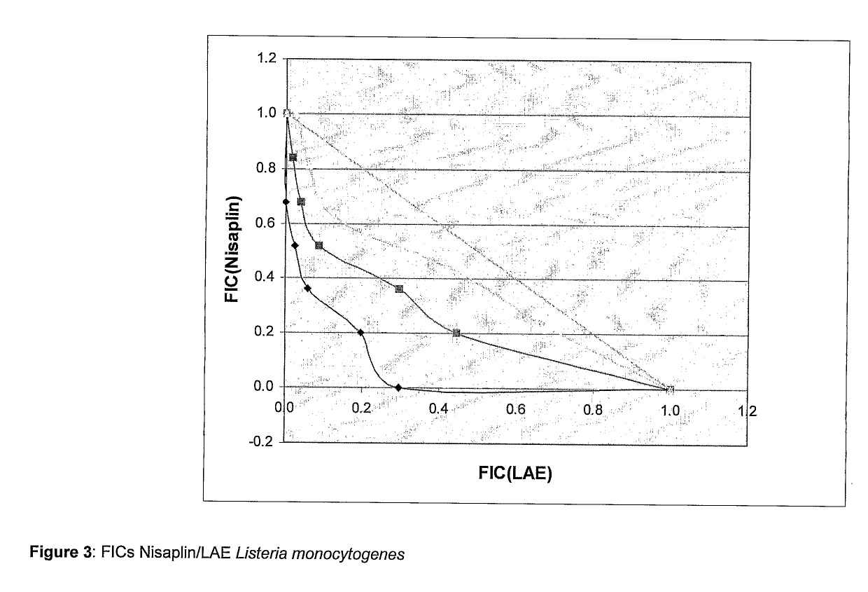

FIG. 3 is a graph;

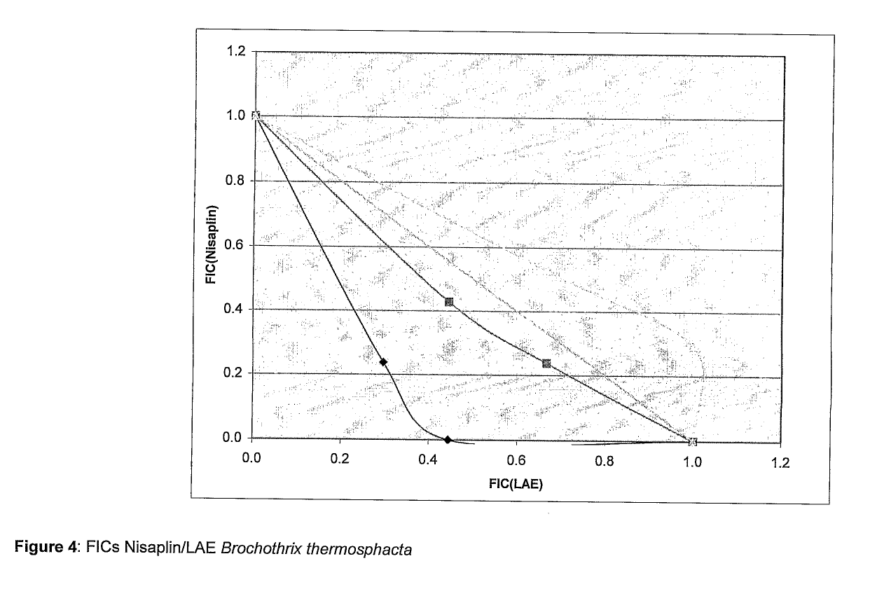

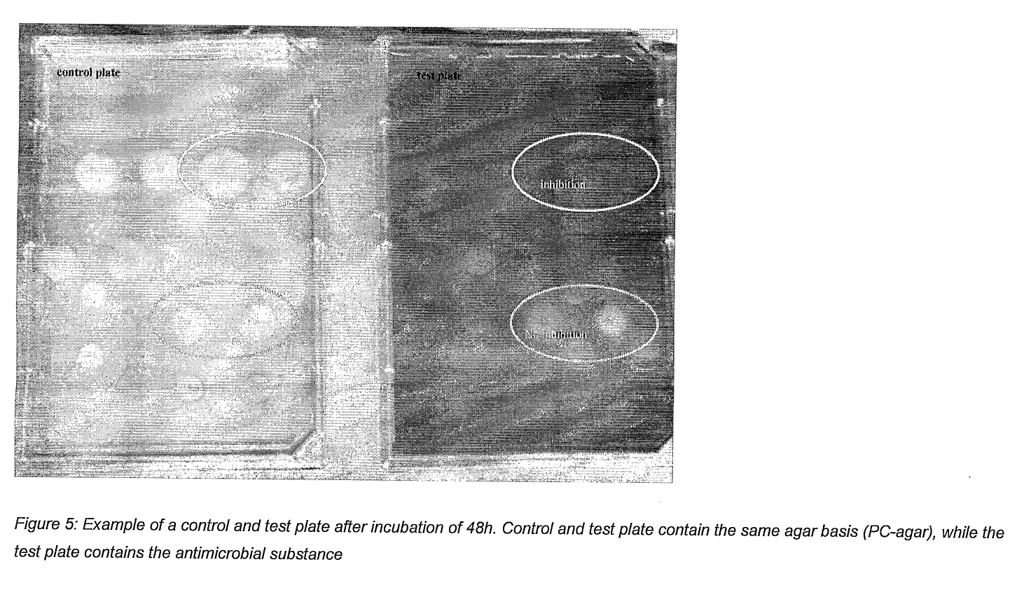

FIG. 4 is a graph;

FIG. 5 is a plate;

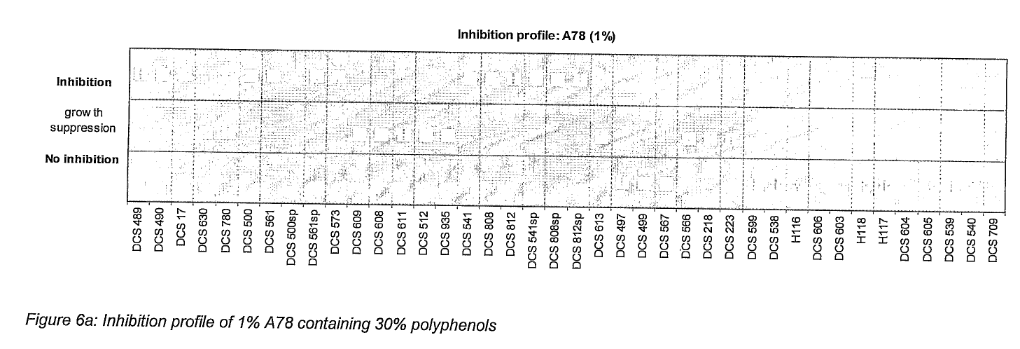

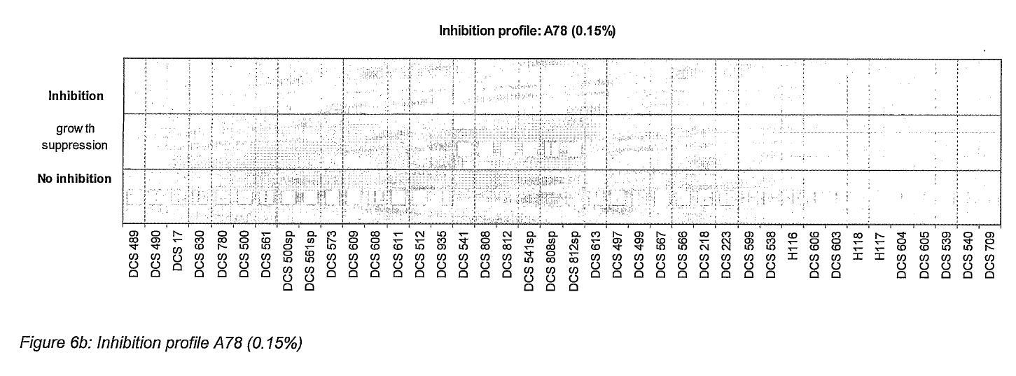

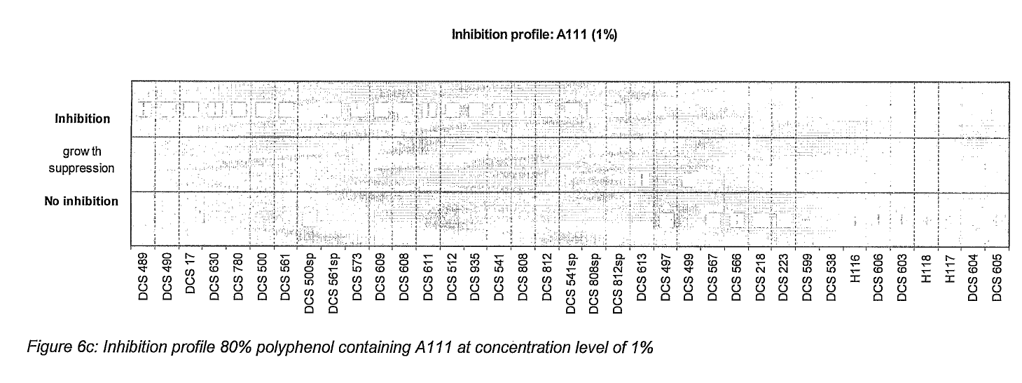

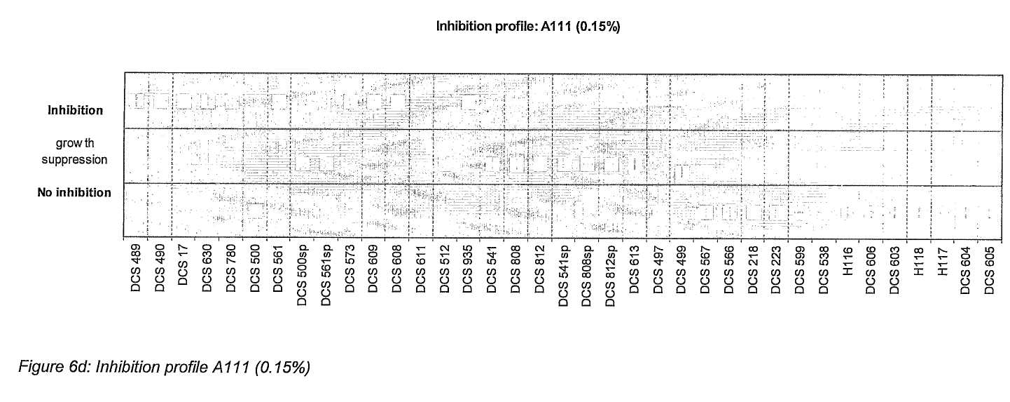

FIG. 6 is a graph;

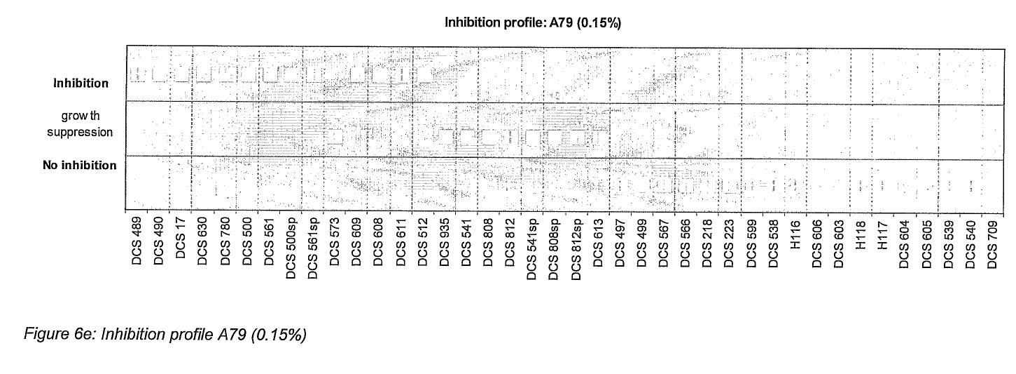

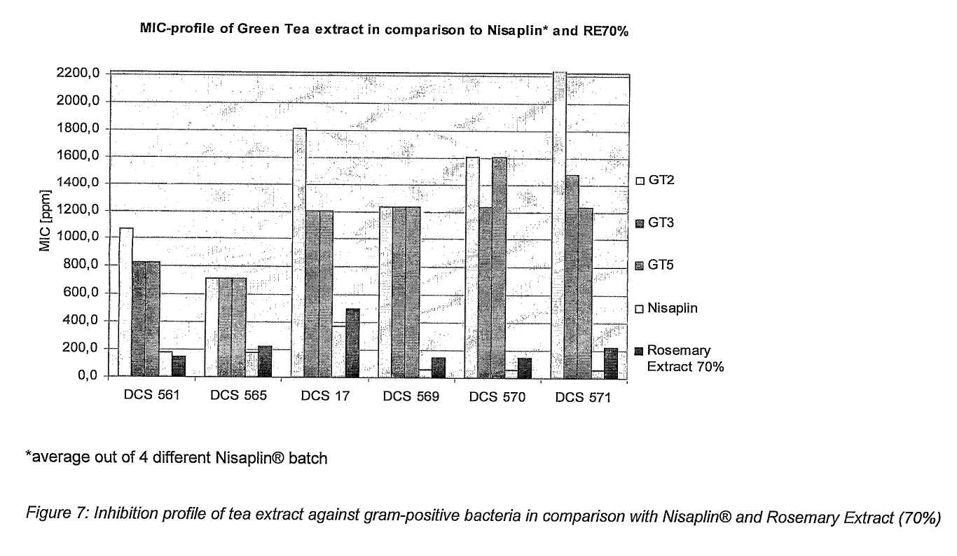

FIG. 7 is a graph;

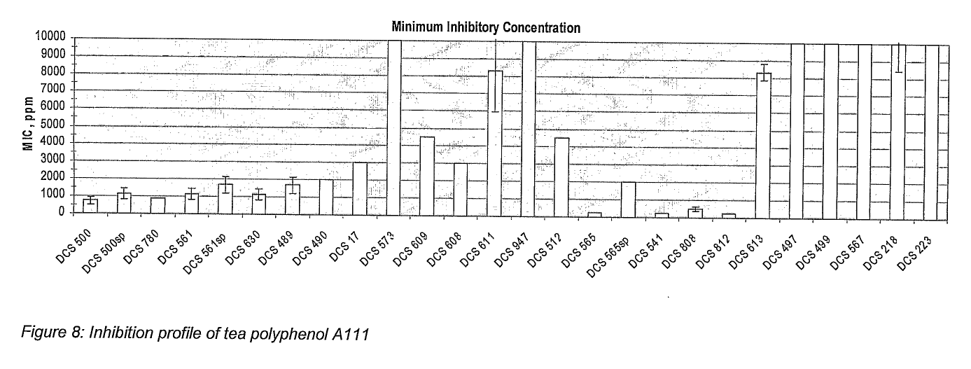

FIG. 8 is a graph; and

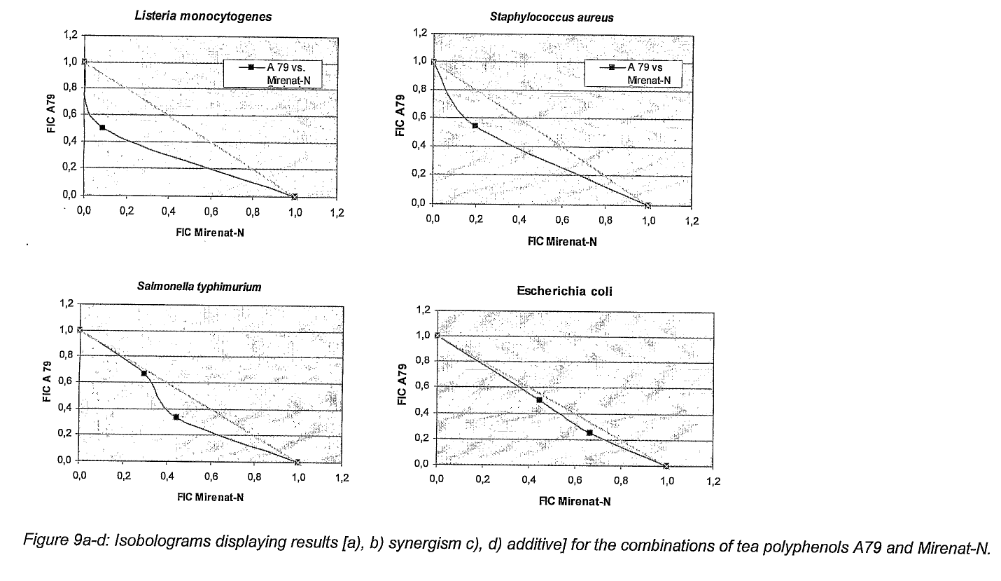

FIG. 9 is a graph.

The present invention will now be described in further detail in the following examples.

### EXAMPLES

Methods Minimal Inhibition Concentration

The Minimal Inhibition Concentration Assay (MIC) is a 96 well liquid based assay developed for a semi-automated assessment system and performed essentially as described in (7). A range of indicator strains (Table 2) is tested for inhibition of growth by a putative anti-microbial substance (AM), using a wide concentration range by performing a ⅔-dilution series from 4.3-166 ppm of LAE (the active component). From an overnight culture 3 ml of each strain was inoculated in one well corresponding to an approximate inoculation density of 10³-10⁴ cells/well. Media used were CASO, MRS and YM (Appendix 1). Strains were incubated at 20° C., 25° C., 37° C. and under aerobic/anaerobic conditions depending on the preferred conditions for the particular strain (Appendix 1). At time zero, after adding the AM, the optical density (O.D.) of the bacterial culture is measured at 620 nm and then again after 24 hours. The increase in O.D. after 24 hours is compared to a growth control sample to estimate whether the substance has a bacteriostatic, increased lag phase or no effect and to determine the MIC. MIC is defined as the lowest concentration of the AM that will inhibit measurable growth. A bacteriostatic effect is defined as the OD₆₂₀ at 24 h being less than or equal to 20% of the growth control. An increased lag phase effect is defined as the OD₆₂₀ at 24 h being less than or equal to 75% of the growth control The MBC (bactericidal effect) is defined as the lowest concentration of AM at which a treated strain shows no growth when transferring it into suitable fresh media (CASO for bacteria, YM for yeast & moulds).

To determine if the solvent of LAE, propylene glycol had antimicrobial activity strains DCS 561, DCS 561 sp, DCS630, DCS 489, DCS 490, DCS 17, DCS 613, DCS 497, DCS 499, DCS 567, DCS 566, DCS 603 and H118 (table 2) were tested in the MIC assay as described above. A ⅔-fold dilution series from 1.2 to 100 ppm of the propylene glycol was used.

The source of strains referred to in the present specification may be identified in the table below

| | | | | Antimicrobial | | |
|---|---|---|---|---|---|---|
| | | | | Concentration [ppm]* | | |
| | | | | increased | | |
| | Strain | No. | Origin | lag phase | MIC | MBC |
| Gram+ | Bacillus cereus | DCS 500 | Isolated from food | 6.5 | 12.1 | 14.6 |
| | Brochothrix thermosphacta | DCS 780 | Isolated from food | 4.3 | 8.1 | 9.7 |
| | Bacillus cereus (spores) | DCS | Spores from DCS 500 | n.d. | n.d. | n.d. |
| | | 500sp | | | | |
| | Bacillus licheniformis | DCS 561 | Isolated from soya powder | 4.3 | 14.6 | 14.6 |
| | Bacillus licheniformis (spores) | DCS | Spores from DCS 561 | 6.5 | 6.5 | 61.5 |
| | | 561sp | | | | |
| | Staphylococcus aureus | DCS 630 | ATCC 29213 | 14.6 | 14.6 | 18.3 |
| | Listeria monocytogenes | DCS 489 | NCTC 12426 | 9.7 | 12.1 | 14.6 |
| | Listeria monocytogenes | DCS 490 | Isolated from food | 8.1 | 12.1 | 12.2 |
| | Listeria innocua | DCS 17 | ATCC 33090 | 5.4 | 12.1 | 21.9 |
| | Lactobacillus fermentum | DCS 573 | Isolated from food | 73.8 | 73.8 | >166 |
| | Lactobacilllus curvatus | DCS 609 | ATCC 25601 | 32.8 | 32.8 | >166 |
| | Lactobacilllus sakei | DCS 608 | DSMZ 15831 | 14.6 | 32.8 | >166 |
| | Lactobacilllus farciminis | DCS 611 | ATCC 29644 | 49.2 | 49.2 | >166 |
| | Leuconostoc spp. | DCS 947 | Isolated from sausage | 32.8 | 32.8 | >166 |
| | Leuconostoc mesenteroides ss | DCS 512 | Isolated from food | 32.8 | 41.0 | >166 |
| | Bacillus weihenstephanensis | DCS 565 | DSMZ 11821 | 10.5 | 14.6 | 14.6 |
| | Bacillus weihenstephanensis | DCS | Spores from DCS 565 | n.d. | n.d. | n.d. |
| | | 565sp | | | | |
| | Clostridium sporogenes | DCS 541 | NCIMB 1793 | 9.7 | 9.7 | 9.7 |
| | Clostridium sporogenes | DCS 808 | Isolated from spoiled cheese | 9.7 | 9.7 | 9.7 |
| | Clostridium sporogenes | DCS 812 | Isolated from Nordic sample | 9.7 | 9.7 | 9.7 |
| | Clostridium sporogenes (spores) | DCS | Spores from DCS 541 | 9.7 | 9.7 | 14.6 |
| | | 541sp | | | | |
| | Clostridium sporogenes (spores) | DCS | Spores from DCS 808 | 8.1 | 8.1 | 18.3 |
| | | 808sp | | | | |
| | Clostridium sporogenes (spores) | DCS | Spores from DCS 812 | 4.3 | 4.3 | 8.1 |
| | | 812sp | | | | |
| | Clostridium algidicarnis | DCS 563 | NCIMB 702929 | 6.5 | 9.7 | 14.9 |
| | Clostridium estertheticum | DCS 568 | NCIMB 12511 | n.d. | n.d. | n.d. |
| Gram− | Hafnia alvei | DCS 613 | DSMZ 30099 | 10.5 | 21.9 | 27.4 |
| | Escherichia coli | DCS 497 | CRA 109 | 18.2 | 21.9 | 21.9 |
| | Pseudomonas fluorescens | DCS 499 | NCIMB 3756 | n.d. | n.d. | n.d. |
| | Klebsiella oxytoca | DCS 567 | ATCC13182 | 18.2 | 32.8 | 41.0 |
| | Citrobacter freundii | DCS 566 | ATCC 8090 | 8.1 | 18.2 | 21.9 |
| | Salmonella typhimurium | DCS 218 | KVL-Copenhagen (P6) | 12.1 | 32.8 | 49.2 |
| | Salmonella typhimurium | DCS 223 | Bio Merieux 1127 | 14.6 | 21.9 | 32.8 |
| Yeast & | Saccharomyces cerevisiae | DCS 599 | CBS 7834 | 14.6 | 14.6 | 14.6 |
| Moulds | Zygosaccharomyces bailii | DCS 538 | CRA 299 | 4.3 | 6.5 | 6.5 |
| | Rhodotorula mucilaginosa | H116 | Internal reference strain | 4.3 | 6.5 | 6.5 |
| | Rhodoturola glutinis | DCS 606 | DSMZ 70398 | n.d. | n.d. | n.d. |
| | Pichia anomala | DCS 603 | ATCC 8168 | 32.8 | 73.8 | 110.7 |
| | Kluyveromyces marxianus | H118 | Internal reference strain | n.d. | n.d. | n.d. |
| | Candida pulcherrima | H117 | Internal reference strain | 6.5 | 9.7 | 14.6 |
| | Candida tropicalis | DCS 604 | DSMZ 1346 | 4.3 | 4.3 | 4.3 |
| | Debaryomyces hansenii | DCS 605 | DSMZ 70238 | 4.3 | 4.3 | 4.3 |
| | Penicillium commune | DCS 539 | Isolated from food | n.d. | n.d. | n.d. |
| | Aspergillus versicolor | DCS 540 | CBS 108959 | n.d. | n.d. | n.d. |
| | Aspergillus parasiticus | DCS 709 | Isolated from food | n.d. | n.d. | n.d. |

DCS: Danisco strain collection
DSMZ: German Collection of Microorganisms and Cell Cultures
NCTC: National Collection of Typed Cultures
NCIMB: National collection of Industrial, Food and Marine Bacteria
CBS: Centraalbureaux voor Schimmelcultures

Methods Fractional Inhibition Concentration

The Fractional Inhibition Concentration (FIC) assay is also a 96 well liquid based assay with a checkerboard titration layout (7, 8), one plate for each strain and sets of concentrations. Concentration ranges of the antimicrobials used for the individual strain are listed in Table 1. Cultivation media can be seen in Appendix 1. O.D. at 620 nm is read at zero hours (at strain addition) and after a 24-hour incubation period. Fractional inhibition concentrations (FIC_{A}=MIC_{A/B}/MIC_{A}) are then calculated to estimate whether there is synergistic, antagonistic or additive effects when combining the two substances. The FICs for the two substances are plotted towards each other in a graph called an isobologram. If the points are below y=x there is a synergistic effect, if points are above there is an antagonistic effect and if the points are on y=x there is an additive effect. Additionally, the FIC index is then calculated as follows: FIC_{index}=FIC_{A}+FIC_{B}, if FIC_{index} is lower than 1 there is synergy, if it is higher than one there is antagony and if it is equal to one there is an additive effect.

FICs were determined for LAE in combination with Nisaplin and Natamax for strains in Table 1A and B.

TABLE 1A
Strains and concentration ranges used in FIC determination with Nisaplin

| | LAE test | |
|---|---|---|
| Strains | range | Nisaplin test range |
| Bacillus licheniformis DCS 561 | 0-50 ppm | 0, 50, 90, 130, 170, 210, 250 ppm |
| Listeria monocytogenes DCS 489 | (⅔ serial dilution) | 0, 50, 90, 130, 170, 210, 250 ppm |
| Brochothrix thermosphacta DCS 780 | | 0, 50, 90, 130, 170, 210, 250 ppm |
| Clostridium sporogenes (sp) DCS 541 | | 0, 50, 90, 130, 170, 210, 250 ppm |
| Escherichia coli DCS 497 | | 0, 250, 450, 650, 850, 1050, 1250 ppm |
| Salmonella typhimurium DCS 218 | | 0, 250, 450, 650, 850, 1050, 1250 ppm |
| Clostridium sporogenes (sp) DCS 812 | | 0, 31, 63, 125, 250, 375, 500 ppm |
| Lactobacillus sakei DCS 608 | | 0, 3, 5, 11, 21, 32, 43 ppm |

TABLE 1B
Strains and concentration ranges used in FIC determination
with Natamax

| | LAE test | Natamax |
|---|---|---|
| Strains | range | test range |
| Saccharomyces cerevisiae DCS 599 | 0-50 ppm or | 0, 0.625, 1.25, 2.5, |
| Zygosaccharomyces bailii DCS 538 | 0-150 ppm | 5, 7.5 and 10 ppm |
| Rhodotorula mucilaginosa H116 | (⅔ serial | and |
| Rhodotorula glutinis DCS 606 | dilution) | 0, 8, 12, 16, |
| Pichia anomala DCS 603 | | 20, 24, 30 ppm |
| Kluyveromyces marxianus H118 | | |
| Candida tropicalis DCS 604 | | |
| Debaromyces hansenii DCS 605 | | |
| Penicillium commune DCS 539 | | |
| Aspergillus parasiticus DCS 709 | | |

Results

Determination of MIC

TABLE 2
minimal inhibitory concentrations

| | | | Antimicrobial | | |
|---|---|---|---|---|---|
| | | | Concentration [ppm]* | | |
| | | | increased lag | | |
| | Strain | No. | phase | MIC | MBC |
| Gram+ | Bacillus cereus | DCS 500 | 6.5 | 12.1 | 14.6 |
| | Brochothrix thermosphacta | DCS 780 | 4.3 | 8.1 | 9.7 |
| | Bacillus cereus (spores) | DCS 500sp | n.d. | n.d. | n.d. |
| | Bacillus licheniformis | DCS 561 | 4.3 | 14.6 | 14.6 |
| | Bacillus licheniformis (spores) | DCS 561sp | 6.5 | 6.5 | 61.5 |
| | Staphylococcus aureus | DCS 630 | 14.6 | 14.6 | 18.3 |
| | Listeria monocytogenes | DCS 489 | 9.7 | 12.1 | 14.6 |
| | Listeria monocytogenes | DCS 490 | 8.1 | 12.1 | 12.2 |
| | Listeria innocua | DCS 17 | 5.4 | 12.1 | 21.9 |
| | Lactobacillus fermentum | DCS 573 | 73.8 | 73.8 | >166 |
| | Lactobacilllus curvatus | DCS 609 | 32.8 | 32.8 | >166 |
| | Lactobacilllus sakei | DCS 608 | 14.6 | 32.8 | >166 |
| | Lactobacilllus farciminis | DCS 611 | 49.2 | 49.2 | >166 |
| | Leuconostoc spp. | DCS 947 | 32.8 | 32.8 | >166 |
| | Leuconostoc mesenteroides ss | DCS 512 | 32.8 | 41.0 | >166 |
| | Bacillus weihenstephanensis | DCS 565 | 10.5 | 14.6 | 14.6 |
| | Bacillus weihenstephanensis | DCS 565sp | n.d. | n.d. | n.d. |
| | Clostridium sporogenes | DCS 541 | 9.7 | 9.7 | 9.7 |
| | Clostridium sporogenes | DCS 808 | 9.7 | 9.7 | 9.7 |
| | Clostridium sporogenes | DCS 812 | 9.7 | 9.7 | 9.7 |
| | Clostridium sporogenes (spores) | DCS 541sp | 9.7 | 9.7 | 14.6 |
| | Clostridium sporogenes (spores) | DCS 808sp | 8.1 | 8.1 | 18.3 |
| | Clostridium sporogenes (spores) | DCS 812sp | 4.3 | 4.3 | 8.1 |
| | Clostridium algidicarnis | DCS 563 | 6.5 | 9.7 | 14.9 |
| | Clostridium estertheticum | DCS 568 | n.d. | n.d. | n.d. |
| Gram− | Hafnia alvei | DCS 613 | 10.5 | 21.9 | 27.4 |
| | Escherichia coli | DCS 497 | 18.2 | 21.9 | 21.9 |
| | Pseudomonas fluorescens | DCS 499 | n.d. | n.d. | n.d. |
| | Klebsiella oxytoca | DCS 567 | 18.2 | 32.8 | 41.0 |
| | Citrobacter freundii | DCS 566 | 8.1 | 18.2 | 21.9 |
| | Salmonella typhimurium | DCS 218 | 12.1 | 32.8 | 49.2 |
| | Salmonella typhimurium | DCS 223 | 14.6 | 21.9 | 32.8 |
| Yeast & | Saccharomyces cerevisiae | DCS 599 | 14.6 | 14.6 | 14.6 |
| Moulds | Zygosaccharomyces bailii | DCS 538 | 4.3 | 6.5 | 6.5 |
| | Rhodotorula mucilaginosa | H116 | 4.3 | 6.5 | 6.5 |
| | Rhodoturola glutinis | DCS 606 | n.d. | n.d. | n.d. |
| | Pichia anomala | DCS 603 | 32.8 | 73.8 | 110.7 |
| | Kluyveromyces marxianus | H118 | n.d. | n.d. | n.d. |
| | Candida pulcherrima | H117 | 6.5 | 9.7 | 14.6 |
| | Candida tropicalis | DCS 604 | 4.3 | 4.3 | 4.3 |
| | Debaryomyces hansenii | DCS 605 | 4.3 | 4.3 | 4.3 |
| | Penicillium commune | DCS 539 | n.d. | n.d. | n.d. |
| | Aspergillus versicolor | DCS 540 | n.d. | n.d. | n.d. |
| | Aspergillus parasiticus | DCS 709 | n.d. | n.d. | n.d. |

*ppm of the active component lauric arginate

In Table 2 and FIG. 1 it is seen that LAE has a very broad range of inhibitory activity with low MICs for the full range of gram positive and negative bacteria and yeasts and moulds tested. MICs range from 4.3 to 73.8 ppm of LAE, which is comparable to nisin MICs. Additionally, it is seen that for most strains, except e.g. the Lactobacillus species, the MBC is very close to the MIC meaning that the effect of LAE is immediate lethality. In addition, for all species an increased lag phase effect is seen at approximately the MIC.

The solvent of LAE, which in the Mirenat-N product is propylene glycol, was tested for antimicrobial activity against a smaller range of both gram positive and negative bacteria and yeasts and moulds. No antimicrobial activity of propylene glycol was observed.

Results FICs with Nisin

TABLE 3
Bacillus licheniformis DCS 561

| Nisaplin | | | | |
|---|---|---|---|---|
| ppm | LAE ppm | FIC_{Nisaplin} | FIC_{LAE} | FIC_{index} |
| 250 | 0 | 1.00 | 0.00 | 1 |
| 210 | 9.9 | 0.84 | 0.20 | 1 |
| 130 | 14.8 | 0.52 | 0.30 | 0.8 |
| 50 | 22.2 | 0.20 | 0.44 | 0.6 |

From Table 3 and FIG. 2 it is clear that at certain combinations of concentrations there is a synergistic effect between nisin and lauric arginate when acting on the gram-positive strain Bacillus licheniformis.

TABLE 4
Listeria monocytogenes DCS 489

| Nisaplin | | | | |
|---|---|---|---|---|
| ppm | LAE ppm | FIC_{Nisaplin} | FIC_{LAE} | FIC_{index} |
| 250 | 0 | 1.00 | 0.00 | 1.00 |
| 210 | 1 | 0.84 | 0.02 | 0.86 |
| 170 | 4 | 0.68 | 0.04 | 0.72 |
| 130 | 5 | 0.52 | 0.09 | 0.61 |
| 90 | 15 | 0.36 | 0.30 | 0.66 |
| 50 | 22 | 0.20 | 0.44 | 0.64 |
| 0 | 50 | 0.00 | 1.00 | 1.00 |

From Table 4 and FIG. 3 it is seen that there is a clear synergistic effect between nisin and lauric arginate when acting on Listeria monocytogenes.

TABLE 5
Brochothrix thermosphacta DCS 780

| Nisaplin | | | | |
|---|---|---|---|---|
| ppm | LAE ppm | FIC_{Nisaplin} | FIC_{LAE} | FIC_{index} |
| 210 | 0 | 1.00 | 0.00 | 1.00 |
| 90 | 22 | 0.43 | 0.44 | 0.87 |
| 50 | 33 | 0.24 | 0.67 | 0.90 |

From Table 5 and FIG. 4 is seen that there is a tendency towards synergy between Nisaplin and LAE for Brochothrix thermosphacta, but more likely the effect observed is additive.

Clostridium sporogenes (spores) DCS 541 and DSC 812 was also tested and a tendency towards an additive effect was observed (results not shown).

FICs with Natamycin

The strains (yeasts and moulds) listed in Table 1B were tested in the combinatory assays between LAE and natamycin.

LAE

Mirenat-N is a 10% w/w solution of lauramide arginine ethyl ester chloride (structure below) in propylene glycol.

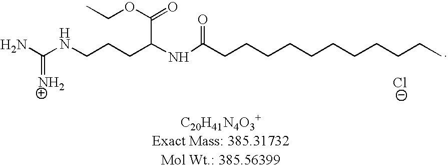

Appendix 1

| | | | Cultivation |
|---|---|---|---|
| Name | Incubation | Full name | medium |
| DCS 500 | 37° C. | Bacillus cereus | CASO |
| DCS 780 | | Brochothrix thermosphacta | CASO |
| DCS 500sp | | Bacillus cereus (spores) | CASO |
| DCS 561 | 37° C. | Bacillus licheniformis | CASO |
| DCS 561sp | | Bacillus licheniformis (spores) | CASO |
| DCS 630 | | Staphylococcus aureus | CASO |
| DCS 489 | 37° C. | Listeria monocytogenes | CASO |
| DCS 490 | | Listeria monocytogenes | CASO |
| DCS 17 | | Listeria innocua | CASO |
| DCS 573 | 37° C. | Lactobacillus fermentum | MRS |
| DCS 609 | | Lactobacilllus curvatus | MRS |
| DCS 608 | | Lactobacilllus sakei | MRS |
| DCS 611 | 37° C. | Lactobacilllus farciminis | MRS |
| DCS 947 | | Leuconostoc spp. | MRS |
| DCS 512 | | Leuconostoc mesenteroides ss | MRS |
| DCS 541 | 37° C. | Clostridium sporogenes | CASO |
| DCS 808 | (anaerob) | Clostridium sporogenes | CASO |
| DCS 812 | | Clostridium sporogenes | CASO |
| DCS 613 | 37° C. | Hafnia alvei | CASO |
| DCS 497 | | Escherichia coli | CASO |
| DCS 499 | | Pseudomonas fluorescens | CASO |
| DCS 567 | 37° C. | Klebsiella oxytoca | CASO |
| DCS 566 | | Citrobacter freundii | CASO |
| DCS 599 | 25° C. | Saccharomyces cerevisiae | YM |
| DCS 538 | | Zygosaccharomyces bailii | YM |
| H116 | | Rhodotorula mucilaginosa | YM |
| DCS 606 | 25° C. | Rhodoturola glutinis | YM |
| DCS 603 | | Pichia anomala | YM |
| H118 | | Kluyveromyces marxianus | YM |
| H117 | 25° C. | Candida pulcherrima | YM |
| DCS 604 | | Candida tropicalis | YM |
| DCS 605 | | Debaryomyces hansenii | YM |
| DCS 539 | 25° C. | Penicillium commune | YM |
| DCS 540 | | Aspergillus versicolor | YM |
| DCS 709 | | Aspergillus parasiticus | YM |
| DCS 565 | 25° C. | Bacillus weihenstephanensis | CASO |
| DCS 565sp | | Bacillus weihenstephanensis | CASO |
| DCS 218 | 37° C. | Salmonella typhimurium | CASO |
| DCS 223 | | Salmonella typhimurium | CASO |
| DCS 541sp | 37° C. | Clostridium sporogenes (spores) | CASO |
| DCS 808sp | (anaerob) | Clostridium sporogenes (spores) | CASO |
| DCS 812sp | | Clostridium sporogenes (spores) | CASO |
| DCS 563 | 20° C. | Clostridium algidicarnis | CASO |
| DCS 568 | (anaerob) | Clostridium estertheticum | CASO |

Tea Extracts

Experimental

TABLE 6
sample list

| | Product description | | | | | | Test range | | | |
|---|---|---|---|---|---|---|---|---|---|---|
| Product | Product | | Physical | | | Company | [ppm] | | | |
| identity | name | Plant name | appearance | Colour | Lot/batch | contact | MIC/MBC | 10000 | 1500 | FIC |
| GT 2 | | Camellia | Fine powder | | E050580-2 | Danisco | x | | | |
| | | sinensis | | | TGP95-SK | (Guardian ™) | | | | |
| GT 3 | tea extract | | | | E050580-3 | | x | | | |
| | | | | | TGP80-SK | | | | | |
| GT 5 | tea extract | | | | E050580-5 | | x | | | |
| | | | | | TGP95-SK | | | | | |
| A 78 | Tea | | | Light | 612061 | Taiyo powe | | x | x | |
| | polyphenols | | | yellow | | | | | | |
| | tea extract | | | | | | | | | |
| | (30%) | | | | | | | | | |
| A 79 | Tea | | | Light | 506133 | | | | x | x |
| | polyphenols | | | yellow | | | | | | |
| | tea extract | | | | | | | | | |
| | (90%) | | | | | | | | | |
| A 111 | Tea | | | | | EUSA | x | x | x | |
| | polyphenols | | | | | Colors | | | | |
| | tea extract | | | | | | | | | |
| | (80%) | | | | | | | | | |

Inhibition Spectrum/Agar-Spot Assay

The tea polyphenols were dissolved or homogenously dispersed in nutrient agar to a final concentration of 1% and 0.15%. 3 μl of overnight bacteria cell suspensions were spotted (in duplicates) on the agar surface. The plates were incubated for 48 h at 37° C. or 25° C. Growth or no growth of the individual strain indicates inhibitory properties of the natural extract.

Minimum Inhibition Concentration Assay

The Minimal Inhibitory Concentration Assay (MIC) is a 96 well liquid based assay developed for automated assessment system. A range of indicator strains (Table 8) is tested for inhibition of growth by the investigated plant extracts, using a wide concentration range (60-3333 ppm) by performing a ⅔-dilution series. From an overnight culture of each strain one well was inoculated corresponding to an approximate inoculation density of 10³-10⁴ cells/well. Media used were CASO, MRS and YM (Appendix 2). Strains were incubated at 20, 25, 37° C. and under aerobic/anaerobic conditions depending on the preferred conditions for the particular strain (Appendix 2).

At time zero, after adding the plant extract, the optical density (O.D.) of the bacterial culture is measured at 620 nm and then again after 24 hours. The increase in O.D. after 24 hours is compared to a growth control sample to estimate whether the substance has a bacteriostatic, increased lag phase or no effect and to determine the MIC. MIC is defined as the lowest concentration of the antimicrobial that will inhibit measurable growth. A bacteriostatic effect is defined as the OD₆₂₀ at 24 h being less than or equal to 20% of the growth control. An increased lag phase effect is defined as the OD₆₂₀ at 24 h being less than or equal to 75% of the growth control.

After incubation and measurement (MIC completed) the MBC (minimum bactericidal concentration) was determined. The MIC-plate is cloned into fresh media—further incubation at optimal growth conditions.

Test Range of Tea Extract Samples:

| | | | | | | | | | | |
|---|---|---|---|---|---|---|---|---|---|---|
| 2222 | 1481 | 988 | 658 | 439 | 293 | 195 | 130 | 87 | 58 | 39 |
| [ppm] | | | | | | | | | | |

Methods Fractional Inhibition Concentration

The Fractional Inhibition Concentration (FIC) assay is also a 96-well liquid based assay with a checkerboard titration layout, one plate for each strain and sets of concentrations. Concentration ranges of the antimicrobials used for the individual strain are listed in Table 7. Cultivation media can be seen in Appendix 2.

O.D. at 620 nm is detected first at zero hours and after a 24-hour incubation period. A strong impact of the test substance on the optical density does not allow the direct use of the OD values for assessing the inhibition activity. Therefore, t=0 h is set up and after a 24 h-incubation this plate is cloned into a new test plate containing cultivation media. The clone is incubated for 24 h and optical density is measured as endpoint detection. Fractional inhibition concentrations (FIC_{A}=MIC_{A/B}/MIC_{A}) are then calculated to estimate whether there is synergistic, antagonistic or additive effects when combining the two substances. FIC was determined for Mirenat-N (LAE) in combination with A79 for 6 strains in Table 7.

The FICs for the two substances are plotted towards each other in a graph called an isobologram. If the points are below y=x there is a synergistic effect, if points are above there is an antagonistic effect and of the points are on y=x there is an additive effect.

TABLE 7
Strains and concentration ranges used in FIC determination with LAE and
Nisaplin ®

| | LAE test | Nisaplin ® | A 79 test | A 111 test |
|---|---|---|---|---|
| Strains | range | test range | range [ppm] | range [ppm] |
| Listeria monocytogenes | 0-55 ppm | 12-666 ppm | 0, 675, 1250, 2500, | 0, 63, 125, 250, |
| DCS 489 | (⅔ serial dilution) | | 5000, 7500, 10000 | 500, 1000, 1500 |
| Staphylococcus aureus | | | | |
| DCS 630 | | | | |
| Bacillus cereus DCS 500 | | | | |
| Bacillus cereus (spores) | | | | |
| DCS 500sp | | | | |
| Clostridium sporogenes | | | | |
| DCS 808 | | | | |
| Clostridium sporogenes | | | | |
| (spores) | | | | |
| DCS 808sp | | | | |
| Escherichia coli DCS 497 | | 60-3333 ppm | | 0, 675, 1250, |
| Salmonella typhimurium | | | | 2500, 5000, |
| DCS 223 | | | | 7500, 10000 |
| Saccharomyces | | Not tested | | Not tested |
| cerevisiae DCS 599 | | | | |
| Kluyveromyces marxianus | | | | |
| H 118 | | | | |

Results

Inhibition Spectrum Determined by a Spot-On-Agar-Assay

FIGS. 6a-e illustrate the observed inhibition activity of the individual plant extracts (with increasing contents of total polyphenols) at concentrations of 1% and 0.15% (w/v). “Inhibition” is defined as when the indicator strain does not grow on the antimicrobial containing agar plate. “Growth suppression” is defined as visible, but not complete growth inhibition, in comparison to the control plate. “No inhibition” is defined as instances where the strain grows comparably on the control and on the test plate (see also FIG. 5).

Using 1% of A78 and A111 showed a broad inhibition spectrum against the assayed gram positive microorganisms. The application of a lower concentration (0.15%) leads in all three cases to the loss of activity.

In this study it was demonstrated that A79, which contains the highest concentration of total polyphenols (90%), was the tea extract with the highest antimicrobial activity. The profiles of FIGS. 6b, 6d and 6e demonstrate the direct correlation of the polyphenol content and the antimicrobial activity.

Gram-negative bacterial growth or inhibit yeasts and moulds were not controlled with the application of A78, A79 and A111.

Minimum Inhibition Concentration Assay

A) Tea Extracts

TABLE 8
Minimum inhibition concentration of three different tea extracts

| | | | MIC - range [ppm] | | |
|---|---|---|---|---|---|
| | Bacterial strain | | MIC | MIC | MIC |
| | Strain | No. | GT 2 | GT 3 | GT 5 |
| Gram+ | Bacillus licheniformis (spores) | DCS 561 | 1070 ± 582 | 823 ± 233 | 823 ± 233 |
| | Bacillus weihenstephanensis | DCS 565 | 714 ± 388 | 714 ± 388 | 714 ± 388 |
| | (spores) | | | | |
| | Listeria innocua | DCS 17 | 1811 ± 712 | 1207 ± 475 | 1207 ± 475 |
| | Lactobacilllus curvatus | DCS 569 | 1235 ± 349 | 1235 ± 349 | 1235 ± 349 |
| | Lactobacilllus curvatus | DCS 570 | 1605 ± 873 | 1235 ± 349 | 1605 ± 873 |
| | Lactobacilllus curvatus | DCS 571 | 2272 ± 786 | 1481 ± 0 | 1235 ± 349 |

In Table 8 and FIG. 7 it is seen that the three tested tea extracts perform comparable to each other with MIC-ranges from 700 ppm to 2200 ppm. GT5 has the least inhibitory activity. When compared to concentrations of Nisaplin® needed to achieve growth inhibition the tea extract is minimum 3-times less effective against Listeria innocua and up to 50-times less active against Lactobacillus curvatus. The same applies for the comparison of tea extract and rosemary extract.

b) Tea Polyphenols

TABLE 9
Minimum bactericidal concentration of A111

| | | | increased | MBC |
|---|---|---|---|---|
| | | | lag phase | A111 |
| | Strain | No. | [ppm] | [ppm] |
| Gram+ | Bacillus cereus | DCS 500 | 731.6 | 731.6 |
| | Bacillus cereus (spores) | DCS 500sp | 1097.3 | 1097.4 |
| | Brochotrix thermosphacta | DCS 780 | 877.9 | 877.9 |
| | Bacillus licheniformis | DCS 561 | 1097.4 | 1097.4 |
| | Bacillus licheniformis | DCS 561sp | 1646.1 | 1646.1 |
| | (spores) | | | |
| | Staphylococcus aureus | DCS 630 | 1097.4 | 1097.4 |
| | Listeria monocytogenes | DCS 489 | 1646.1 | 1646.1 |
| | Listeria monocytogenes | DCS 490 | 1975.3 | 1975.3 |
| | Listeria innocua | DCS 17 | 2963.0 | 2963.0 |
| | Lactobacillus fermentum | DCS 573 | >10000 | >10000 |
| | Lactobacilllus curvatus | DCS 609 | 4444.4 | 4444.4 |
| | Lactobacilllus sakei | DCS 608 | 2963.0 | 2963.0 |
| | Lactobacilllus farciminis | DCS 611 | 8333.3 | 8333.3 |
| | Leuconostoc | DCS 947 | >10000 | >10000 |
| | mesenteroides ss | | | |
| | Leuconostoc | DCS 512 | 4444.4 | 4444.4 |
| | mesenteroides ss | | | |
| | Bacillus | DCS 565 | 260.1 | 260.1 |
| | weihenstephanensis | | | |
| | Bacillus | DCS 565sp | 390.2 | 1975.3 |
| | weihenstephanensis | | | |
| | Clostridium sporogenes | DCS 541 | 260.1 | 260.1 |
| | Clostridium sporogenes | DCS 808 | 487.7 | 487.7 |
| | Clostridium sporogenes | DCS 812 | 260.1 | 260.1 |
| Gram− | Hafnia alvei | DCS 613 | 1646.1 | 8333.3 |
| | Escherichia coli | DCS 497 | >10000 | >10000 |
| | Pseudomonas fluorescens | DCS 499 | 6666.0 | 10000.0 |
| | Klebsiella oxytoca | DCS 567 | >10000 | >10000 |
| | Salmonella typhimurium | DCS 218 | 5555.6 | >10000 |
| | Salmonella typhimurium | DCS 223 | >10000 | >10000 |

The tea polyphenol A111 and the three tea extracts show comparable MIC towards the test organism DCS 561. Concentrations needed to inhibit Listeria innocua (DCS 17) are approximately 1.5-times higher using A111 in comparison with the extracts. However it needs to be noted that MBC are compared to MIC, which could lead to these differences. (Modifications on the methods as well as different locations when executing the assays lead to different set-up and therefore to the detection of MIC for the green tea extracts and the detection of MBCs for the green tea polyphenols)

Inhibition concentration against spoilage bacteria such as the tested Lactobacillus strains is up to ten-times higher than towards Bacillus spp.

Performing a liquid based inhibition assay confirmed that the plant extracts are not able to inhibit gram-negative bacterial growth. Yeasts and moulds were not tested.

Combinatory Assay

In an effort to explore the possibility of producing new blends with natural plant extracts and other antimicrobials, a set of FIC experiments was carried out in which different concentrations of LAE or Nisaplin® were mixed with a tea polyphenol (A79). The results can be seen in Table 10.

TABLE 10
Interaction of tea polyphenols (A111 & A79) with Mirenat-N or Nisaplin ®

| | | A 111 | | | | | | | | A 79 | | | |
|---|---|---|---|---|---|---|---|---|---|---|---|---|---|
| Strain | | Nisaplin ® | | | | | Nisaplin ® | | | Mirenat-N | | | |
| ID | Species | MIC_{Nisaplin ®} | | Effects | MIC_{A111} | | MIC_{Nisaplin ®} | | Effects | MIC_{Mirenat-N} | Effects | MIC_{A79} | |
| DCS | Listeria mono- | 200 | ppm | Additive (A) | 2000 | ppm | 200 | ppm | Additive | 24 ppm | (weak) | 2500 | ppm |
| 489 | cytogenes | | | | | | | | (A) | | Synergism | | |
| DCS | Staphylococcus | 500 | ppm | No | 1500 | ppm | 500 | ppm | No | 16 ppm | (weak) | 1250 | ppm |
| 630 | aureus | | | Interaction | | | | | Interaction | | Synergism | | |
| DCS | Bacillus cereus | 17 | ppm | No | 1000 | ppm | 17 | ppm | Additive | n.t. | n.t. | 1500 | ppm |
| 500 | | | | Interaction | | | | | (A) | | | | |
| DCS | Bacillus cereus | 13 | ppm | No | 1000 | ppm | 13 | ppm | Additive | n.t. | n.t. | 1000 | ppm |
| 500sp | (spores) | | | Interaction | | | | | (A) | | | | |
| DCS | Clostridium | 200 | ppm | Additive (A) | 500 | ppm | 200 | ppm | Additive | n.t. | n.t. | 7500 | ppm |
| 800 | sporogenes | | | | | | | | (A) | | | | |
| DCS | Clostridium | 30 | ppm | Additive (A) | 400 | ppm | 30 | ppm | Additive | n.t. | n.t. | 2500 | ppm |
| 808sp | sporogenes | | | | | | | | (A) | | | | |
| | (spores) | | | | | | | | | | | | |
| DCS | Salmonella | >3333 | ppm | No | >10000 | ppm | >3333 | ppm | No | 37 ppm | Additive | >10000 | ppm |
| 223 | typhimurium | | | Interaction | | | | | Interaction | | (A) | | |
| DCS | Escherichia coli | >3333 | ppm | No | >10000 | ppm | >3333 | ppm | No | 37 ppm | Additive | >10000 | ppm |
| 497 | | | | Interaction | | | | | Interaction | | (A) | | |

| | | | | | | | | | | |
|---|---|---|---|---|---|---|---|---|---|---|
| DCS | Saccharomyces | n.t. | n.t. | n.t. | n.t. | n.t. | 24 ppm | No | >10000 | ppm |
| 599 | cerevisiae | | | | | | | Interaction | | |
| H 118 | Kluyveromyces | n.t. | n.t. | n.t. | n.t. | n.t. | 7 ppm | No | >10000 | ppm |
| | marxianus | | | | | | | Interaction | | |

n.t.: not tested

The Mirenat-N in combination with the tea polyphenol A 79 showed uniform degrees of activity within the different groups of microorganisms examined. As expected the gram-positive bacteria are more sensitive to the exposure of the blend than the Gram-negatives and the yeast, respectively as can be seen with the lower MICs of the individual compounds in Table 10.

Synergism was observed between LAE and A79 against the test organisms Listeria monocytogenes (FIG. 9a) and Staphylococcus aureus (FIG. 9b). The combination of Mirenat-N and A79 (FIG. 9c/d) showed additive effects against the two tested gram-negative bacteria Salmonella typhimurium and Escherichia coli.

When combining LAE with A 79 against the yeast Saccharomyces cerevisae and Kluyveromyces marxianus as no interaction could be observed.

The performed combinatory assay of A 111 and Nisaplin® showed additive effects when tested against Listeria monocytogenes and Clostridium sporogenes. The same effect could be observed for the Nisaplin®/A 79-blend. No beneficial interaction was observed for the indicator strains Escherichia coli and Salmonella typhimurium. Neither Nisaplin® or the extracts alone nor the tested combination could inhibit bacterial growth of the Gram-negatives. Combining Nisaplin® with the tea polyphenols is not enhancing the antimicrobial activity of the individual compounds towards Staphylococcus aureus.

The modes of interaction of LAE with A79 are presented for Listeria monocytogenes, Staphylococcus aureus, Salmonella typhimurium and Escherichia coli as FIC isobolograms in FIG. 9a-d. A degree of synergism or antagonism observed, is indicated by the curve of the line away from the theoretically additive line (below: synergy; above: antagonism.

Hops, Grape Seed, Grape Skin & Uva Ursi

Experimental

The preservative properties of different plant extracts, was evaluated by determination of MIC using a broth micro-dilution method against bacterial and fungal microorganism. Of the herbs and spices officially recognized as useful for food ingredients, only a handful has demonstrated significant antimicrobial activity. In many cases, concentrations of the antimicrobial compounds in herbs and spices are too low to be used effectively without adverse effects on the sensory characteristics of a food product.

Investigated Strains

A collection of test organisms (See Appendix 2), including bacterial strains, both Gram-positive (spore and vegetative forms) as well as Gram-negative, and fungal strains were used to assess the anti-microbial properties of the test samples. The strains were chosen to represent several major groups. All species used, with the exception of Clostridia spp. were aerobic.

Plant Materials

The plant extracts used in this research were obtained from commercial sources (table 11). All samples were stored at room temperature in the dark prior testing.

Many of the plant extracts are immiscible in aqueous buffers used in bactericidal assays. It was noted that during suspension preparation that some extracts separated more slowly than others. Constantly shaking until the time of use, as part of the sample preparation to suspend the water-insolubility, was chosen as a simple method approximating what a processor can do without further equipment. The suspensions of plant extracts showed often high colour impacts to the media.

TABLE 11
Tested plant extracts

| Product | | | | Application | | |
|---|---|---|---|---|---|---|
| ID | Product name | Plant name | Colour | Functionality | Products | Company |

Hops Extract

| | | | | | | |
|---|---|---|---|---|---|---|
| A105 | NOVA (5% hop acid, | Humulus | Yellow | Reduces | Beverage | Femto |
| | tetrahydroisohumulone) | lupulus | | volatile acid | | technologies |
| | | (Cannabaceae) | | formation, | | |
| | | | | Antimicrobial | | |
| A106 | Lupulite (30% hops | Humulus | Yellow | Antimicrobial | Beverage | Femto |
| | extract) | lupulus | | | | technologies |
| | | (Cannabaceae) | | | | |

Fruit Extract

| | | | | |
|---|---|---|---|---|
| A81 | Uva Ursi 20% | Arctostaphylos | Brown | Nutrafur |
| | | uva ursi | | |
| | | (Ericaceae) | | |

Grape Extract

| | | | | | | |
|---|---|---|---|---|---|---|
| A70 | Grap′Active ® White H | Vitis vinifera | Light | Antioxidant | Dairy, | Ferco |
| | (White grape seed | (Vitaceae) | brown | | fruits, | |
| | (80%)) | | | | dietetic | |
| A73 | Grap′Active ® Seed M | Vitis vinifera | Tawny | Antioxidant | Dairy, | Ferco |
| | (Grape seed extract | (Vitaceae) | | | fruits, | |
| | (90%)) | | | | dietetic | |
| A68 | Grap′Active ® Red H | Vitis vinifera | Dark | Antioxidant, | Dairy, | Ferco |
| | (Red grape skin (80%)) | (Vitaceae) | mauve | colouring | fruits, | |
| | | | | | dietetic | |

Results and Discussion

Anti-Microbial Activity

Qualitative results, (‘+’ inhibition, ‘(+)’ growth suppression and ‘−’ no inhibition) were obtained by the pre-screen and are summarised in table 14. Most of the plant extracts show good antibacterial activity against Gram-positives. The bacterial strains belonging to the group of Gram-negatives were not inhibited but some were influenced by the presence of the plant extracts.

Due to promising antimicrobial activities against the Gram-positive bacteria an MIC-assay was performed using concentration ranges from 5 to 2000 ppm for the hops extracts and 260 ppm to 10000 ppm for the grape extracts and the uva ursi extract.

MICs for hops extract range from 5 to 60 ppm of both hops extracts, while as A 105 (tetrahydro-isohumulone) performs slightly better than A106 (isohumulone) for some of the Lactic acid bacteria and Listeria strains. The MIC detected are below the MIC of Nisaplin®, which points out the potential use of the Hops extracts as natural antimicrobial.

Extracts obtained from grape seeds, which are by-products of the wine and juice industries, contain large quantities of monomeric phenolic compounds and dimeric, trimeric and tetrameric pro-cyanidins, and have been reported to have many favourable effects on human health used as natural antioxidants. The comparison of grape seed (A73/A70) and grape skin (A68) extracts demonstrated stronger inhibition activity for the grape seed extracts. A correlation between the content of polyphenols and the inhibition activity was shown for A73 and A70. The higher polyphenol content in A73 resulted in a slightly better inhibition activity.

The anti-bacterial activities of the plant extracts presented are in general agreement with previously reported studies. All the bacterial strains demonstrated some degree of sensitivity to the plant extract tested. This was shown in a spot-agar test with the application of 10000 ppm of different plant extracts (hops extract “NOVA” (A105); hops extract “Lupulite” (A106), grape skin extract (A68); grape seed extract (A70&A73) Uva Ursi extract (A81)). It is seen that the hops extracts have a very broad spectrum of inhibitory activity with low MICs for the full range of Gram-positive bacteria tested. MICs range from 5 to 60 ppm of both tested hops extracts, while as A105 performs slightly better than A106 for some of the Lactic acid bacteria and Listeria strains. The MIC detected are below the MIC of Nisaplin®, which points out the potential use of the Hops extracts as natural antimicrobial.

The comparison of grape seed (A73/A70) and grape skin (A68) extracts demonstrated stronger inhibition activity for the grape seed extracts. A correlation between the content of polyphenols and the inhibition activity was shown for A73 and A70. The higher polyphenol content in A73 resulted in a slightly better inhibition activity. The observation of the capacity of plant extracts as natural compounds to inhibit food pathogens and food spoilage, singly and in combination with other antimicrobials, which was demonstrated in several combinatory assay e.g. additive effect against Listeria monocytogenes and Staphylococcus aureus was seen when the hops, was combined with LAE. A trend for synergy was seen for the blends with grape skin (A68) and grape seed (A73) extract and the uva ursi extracts (A81), respectively, when tested against Listeria monocytogenes.

TABLE 12
Qualitative (‘+’ inhibition, ‘(+)’ growth suppression and ‘−’ no inhibition) inhibition spectrum of
the different plant extracts, grouped by their origin, using 1% (w/v) in a spot-agar test.
(“/” not tested or no growth in the growth control - see also Appendix 2)

| DCS | | | | | | | | | | | | | |
|---|---|---|---|---|---|---|---|---|---|---|---|---|---|
| 500 | 500s | 780 | 561 | 561s | 630 | 489 | 490 | 17 | 935 | 609 | 611 | 573 | 608 |

Hops Extracts

| | | | | | | | | | | | | | | |
|---|---|---|---|---|---|---|---|---|---|---|---|---|---|---|
| A105 | + | + | + | + | + | + | + | + | + | + | + | + | + | + |
| A106 | + | + | + | + | + | + | + | + | + | + | + | + | + | + |

Fruit Extracts

| | | | | | | | | | | | | | | |
|---|---|---|---|---|---|---|---|---|---|---|---|---|---|---|
| A81 | + | + | + | + | + | + | + | + | + | (+) | (+) | (+) | (+) | + |

Grape Extracts

| | | | | | | | | | | | | | | |
|---|---|---|---|---|---|---|---|---|---|---|---|---|---|---|
| A70 | + | + | + | + | + | + | + | + | + | + | (+) | (+) | + | + |
| A73 | + | + | + | + | + | + | + | + | + | + | (+) | + | (+) | + |
| A68 | + | + | + | + | + | + | + | + | + | + | + | + | + | + |

| DCS | | | | | | | | | | | | |
|---|---|---|---|---|---|---|---|---|---|---|---|---|
| 512 | 541 | 808 | 812 | 541s | 808s | 812s | 613 | 497 | 499 | 458 | 567 | 566 |

Hops Extracts

| | | | | | | | | | | | | | |
|---|---|---|---|---|---|---|---|---|---|---|---|---|---|
| A105 | + | + | + | + | + | + | + | − | − | − | / | − | − |
| A106 | + | + | + | + | + | + | + | − | − | (+) | / | − | (+) |

Fruit Extracts

| | | | | | | | | | | | | | |
|---|---|---|---|---|---|---|---|---|---|---|---|---|---|
| A81 | + | + | + | + | + | + | + | + | − | − | / | − | (+) |

Grape Extracts

| | | | | | | | | | | | | | |
|---|---|---|---|---|---|---|---|---|---|---|---|---|---|
| A70 | (+) | + | + | + | + | + | + | (+) | − | − | / | − | − |
| A73 | + | + | + | (+) | + | + | + | (+) | (+) | − | / | − | − |
| A68 | + | + | + | + | + | + | + | (+) | − | − | / | − | − |

| | DCS | | | | | | | | | | | |
|---|---|---|---|---|---|---|---|---|---|---|---|---|
| | 218 | 223 | 599 | 538 | 1087 | 606 | 603 | 1089 | 1088 | 604 | 605 | 539 |
| Hops Extracts | | | | | | | | | | | | |
| A105 | − | − | − | − | − | (+) | − | − | − | − | − | − |
| A106 | (+) | (+) | − | − | − | (+) | − | − | − | − | (+) | − |
| Fruit Extracts | | | | | | | | | | | | |
| A81 | + | + | − | − | + | + | − | + | − | − | − | − |
| Grape Extracts | | | | | | | | | | | | |
| A70 | − | − | − | − | − | − | − | − | − | − | − | − |
| A73 | / | / | − | − | − | − | − | − | − | − | − | − |
| A68 | − | − | − | − | − | − | − | − | − | − | − | − |

Combinatory Assay

In an effort to explore the possibility of producing new blends with natural plant extracts and Mirenat-N (A15) (LAE—as shown herein), a set of FIC experiments was carried out in which different concentration combinations of the hops extracts (A105; A106), grape extract (A68, A70; A73) and uva ursi extract (A81) (table 13) were tested for relevant indicator strains. LAE has previously been shown to have a unique broad range of anti-microbial activity, and it has been shown to maintain this activity over a pH range from 3 to 7.

The results can be seen in table 14.

The fractional inhibition concentration (FIC) assay is a 96-well, liquid-based assay with a checkerboard titration layout, that allows varying concentrations of each antimicrobial along the different axes (one plate for each strain and sets of concentrations). The OD at 620 nm is measured at zero hours (at strain addition (10³-10⁴ cfu/ml)) and after a 24-hour incubation period. Due to high impacts of the extracts on the media the plate was cloned into fresh media (CASO-broth; pH 6.0) and further incubated (24 h). Fractional inhibition concentrations (FIC_{A}=MIC_{A/B}/MIC_{A}) were then used to estimate the interaction when combining the two substances (synergistic, antagonistic or additive effects, (no interaction), respectively).

The FIC index is then calculated as follows: FIC_{index}=FIC_{A}+FIC_{B}. An index between 0 and 0.9 is defined as synergy. FIC values between 0.9 and 1.1 are defined as additive effect. Antagony can be concluded from an FIC_{index} greater than 1.1.

TABLE 13
Investigated concentrations of plant extracts Mirenat-N (A15)

| | [ppm] | Natural plant extract test range [ppm] | | | | |
|---|---|---|---|---|---|---|
| | LAE | Grape Extracts | | Hops Extract | | |
| Strain | A15 | A68 | A73 | A105 | A106 | A81 |
| DCS | 1-55* | 10000; | 10000; | 100; | 50; | 10000; |
| 489 | | 7500; | 7500; | 50; | 25; | 7500; |
| DCS | | 5000; | 5000; | 25; | 15; | 5000; |
| 630 | | 2500; | 2500; | 15; | 10; | 2500; |
| | | 1250; | 1250; | 10; | 5; | 1250; |
| | | 675; | 675; | 5; | 2.5; | 675; |
| | | 0 | 0 | 2.5; | 1.25; | 0 |
| | | | | 0 | 0 | |
| DCS | | | | 2000; | 2000; | |
| 497 | | | | 1000 | 1000; | |
| DCS | | | | 500; | 500; | |
| 218 | | | | 250; | 250; | |
| DCS | 3-166* | | | 125; | 125; | |
| 599 | | | | 63; | 63; | |
| DCS | | | | 0 | 0 | |
| 1089 | | | | | | |

*2/3 serial dilution

The LAE and the hops extract (A105; A106) in combination showed additive effects against the Gram-positive test organism.

A trend for synergy was seen for the blends with grape skin (A68) and grape seed (A73) extract when tested against Listeria monocytogenes. Combining LAE with A70 another grape seed extract additive effects of the anti-microbial activity of the individual compounds could be observed. The different behaviour of the two grape seed extracts could be in correlation with the different polyphenol content of the extracts.

Furthermore, beneficial interaction was observed for the blend of LAE and uva ursi (A81) when tested against the Gram-positive indicator strains.

TABLE 14
Interaction of plant extracts with Mirenat-N

| | MIC_{A15} | MIC_{A105} | FIC_{A15} | FIC_{A105} | FIC_{index} | |
|---|---|---|---|---|---|---|
| DCS 489 | 16 | 0 | 1 | 0 | 1 | Additive |
| (Hops | 11 | 15 | 0.67 | 0.3 | 0.97 | |
| Extract) | 0 | 50 | 0 | 1 | 1 | |
| DCS 630 | 16 | 0 | 1 | 0 | 1 | Additive |
| (Hops | 11 | 10 | 0.67 | 0.4 | 1.07 | |
| Extract) | 5 | 15 | 0.3 | 0.6 | 0.9 | |
| | 0 | 25 | 0 | 1 | 1 | |
| | MIC_{A15} | MIC_{A68} | FIC_{A15} | FIC_{A68} | FIC_{index} | |
| DCS 489 | 24 | 0 | 1 | 0 | 1 | Synergy |
| (Hops | 16 | 2500 | 0.67 | 0.33 | 1 | |
| Extract) | 2 | 5000 | 0.09 | 0.67 | 0.76 | |
| | 0 | 7500 | 0 | 1 | 1 | |
| DCS 630 | 16 | 0 | 1 | 0 | 1 | Additive |
| (Hops | 11 | 675 | 0.67 | 0.54 | 1.2 | |
| Extract) | 0 | 1250 | 0 | 1 | 1 | |
| | MIC_{A15} | MIC_{A70} | FIC_{A15} | FIC_{A70} | FIC_{index} | |
| DCS 489 | 24 | 0 | 1 | 0 | 1 | Additive |
| (Grape | 16 | 2500 | 0.67 | 0.33 | 1 | |
| Extract) | 11 | 5000 | 0.44 | 0.67 | 1.1 | |
| | 0 | 7500 | 0 | 1 | 1 | |
| DCS 63 | 16 | 0 | 1 | 0 | 1 | Additive |
| (Grape | 11 | 675 | 0.67 | 0.27 | 0.94 | |
| Extract 0 | 7 | 1250 | 0.44 | 0.5 | 0.94 | |
| | 0 | 2500 | 0 | 1 | 1 | |
| | MIC_{A15} | MIC_{A73} | FIC_{A15} | FIC_{A73} | FIC_{index} | |
| DCS 489 | 24 | 0 | 1 | 0 | 1 | Synergy |
| (Grape | 16 | 1250 | 0.67 | 0.25 | 0.92 | |
| Extract | 7 | 2500 | 0.3 | 0.5 | 0.8 | |
| | 0 | 5000 | 0 | 1 | 1 | |
| DCS 630 | 24 | 0 | 1 | 0 | 1 | Additive |
| (Grape | 7 | 675 | 0.3 | 0.54 | 0.84 | |
| Extract | 0 | 1250 | 0 | 1 | 1 | |
| | MIC_{A15} | MIC_{A81} | FIC_{A15} | FIC_{A81} | FIC_{index} | |
| DCS 489 | 24 | 0 | 1 | 0 | 1 | Synergy |
| (Uva Ursi | 16 | 675 | 0.67 | 0.14 | 0.81 | |
| extract) | 5 | 1250 | 0.13 | 0.25 | 0.38 | |
| | 1 | 2500 | 0.04 | 0.5 | 0.54 | |
| | 0 | 5000 | 0 | 1 | 1 | |
| DCS 630 | 24 | 0 | 1 | 0 | 1 | Synergy |
| (Uva Ursi | 7 | 675 | 0.3 | 0.54 | 0.84 | |
| extract) | 0 | 1250 | 0 | 1 | 1 | |

Conclusion

The plant extracts clearly demonstrate antibacterial properties. These activities suggest potential use as chemotherapeutic agents, food preserving agents and disinfectants. The tested plant products appear to be effective against a wide spectrum of microorganisms, both pathogenic and non-pathogenic. Especially strong antimicrobial activity regarding low MICs was observed with the hops extract.

The effects identified between LAE and some plant extracts could enable the use of lower amounts of both compounds for an effective food preservation strategy.

Appendix 2

| | Strain | No. | Media |
|---|---|---|---|
| Gram+ | Bacillus cereus | DCS 500 | |
| | Bacillus cereus (spores) | DCS 500sp | |
| | Brochothrix thermosphacta | DCS 780 | CASO- |
| | Bacillus licheniformis | DCS 561 | broth/ |
| | Bacillus licheniformis (spores) | DCS 561sp | PC-Agar |
| | Staphylococcus aureus | DCS 630 | (VWR: |
| | Listeria monocytogenes | DCS 489 | 620707A |
| | Listeria monocytogenes | DCS 490 | ES) |
| | Listeria innocua | DCS 17 | |
| | Bacillus weihenstephanensis | DCS 565 | CASO- |
| | Bacillus weihenstephanensis | DCS 565sp | broth |
| | (spores) | | |
| | Lactobacilllus carnosum | DCS 935 | |
| | Lactobacilllus curvatus | DCS 569 | MRS- |
| | Lactobacilllus curvatus | DCS 570 | Agar/- |
| | Lactobacilllus curvatus | DCS 571 | broth |
| | Lactobacilllus curvatus | DCS 609 | (VWR: |
| | Lactobacilllus farciminis | DCS 611 | 6217578A |
| | Lactobacillus fermentum | DCS 573 | ES) |
| | Lactobacillus sakei | DCS 608 | 37° C. |
| | Leuconostoc mesenteroides ss | DCS 512 | |
| | Clostridium sporogenes | DCS 541 | CASO- |
| | Clostridium sporogenes | DCS 808 | broth/ |
| | Clostridium sporogenes | DCS 812 | PC-Agar |
| | Clostridium sporogenes (spores) | DCS 541sp | (VWR: |
| | Clostridium sporogenes (spores) | DCS 808sp | 620707A |
| | Clostridium sporogenes (spores) | DCS 812sp | ES) |
| | | | Anaerob |
| | | | 37° C. |
| Gram− | Hafnia alvei | DCS 613 | |
| | Escherichia coli | DCS 497 | CASO- |
| | Pseudomonas fluorescens | DCS 499 | broth/ |
| | Pseudomonas putida | DCS 458 | PC-Agar |
| | Klebsiella oxytoca | DCS 567 | (VWR) |
| | Citrobacter freundii | DCS 566 | 37° C. |
| | Salmonella typhimurium | DCS 218 | |
| | Salmonella typhimurium | DCS 223 | |
| Yeast & | Saccharomyces cerevisiae | DCS 599 | |
| Moulds | Zygosaccharomyces bailii | DCS 538 | |
| | Rhodotorula mucilaginosa | DCS 1087 | |
| | | (H116) | |
| | Rhodoturola glutinis | DCS 606 | YGC- |
| | Pichia anomala | DCS 603 | Agar/- |
| | Kluyveromyces marxianus | DCS 1089 | broth |
| | | (H118) | (heipha: |
| | Candida pulcherrima | DCS 1088 | 545200) |
| | | (H117) | 25° C. |
| | Candida tropicalis | DCS 604 | |
| | Debaryomyces hansenii | DCS 605 | |
| | Penicillium commune | DCS 539 | |

### REFERENCES

1. Bakal G and Diaz A. 2005. The lowdown on lauric arginate. Food quality magazine. February/March 2005.

2. WO 96/21642, WO 01/94292, WO 03/064669, WO 02/087328, WO 03/034842, WO 03/094638, WO 03/013453, WO 03/013454, WO 03/043593, WO2007014580, WO2007005410, WO2006084553

3. http://wvvw.vedeqsa.com/main.htm

4. Luchansky J B, Call J E, Hristova B, rumery L , Yoder L, Oser A. 2005. Viability of Listeria monocytogenes on commercially-prepared hams surface treated with acidic calcium sulfate and lauric arginate and stored at 4° C. Meat Sci. 71: 92-99.

5. Rodriguez E, Seguer J, Rocabayera X, Manresa A. 2004. Cellular effects of monohydrochloride of L-arginine N^{□}-lauroyl ethylester (LAE) on exposure to Salmonella typhimurium and Staphylococcus aureus. J Appl Microbiol 96: 903-912.

6. Davidson P M, Sofos J N, Branen A L. 2005. Antimicrobials in Food. CRC, 3^{rd} edition. 7. Davidson, P M, and Parish M E. 1989. Methods for testing the efficacy of food antimicrobials. Food Technol. 43:148-155.

8. Olasupo N A, Fitzgerald D J, Narbad A, Gasson M J. 2004. Inhibition of Bacillus subtilis and Listeria innocua by Nisin in combination with some naturally occurring organic compounds. J Food Protect 67(3): 596-600.

9. GRAS notification no. 000164: http://www.cfsan.fda.gov/˜rdb/opag164a.html and http://www.cfsan.fda.qov/˜rdb/opa-g164.html

10. FISI Directive 7120.1: http://vvww.fsis.usda.gov/OPPDE/rdad/FSISDirectives/7120.1Amend_{—}5.pdf

11. CHENG-CHUN, C.; LON-LEU, L. KING-THOM, C., 1999, Antimicrobial activity of tea as affected by the degree of fermentation and manufacturing season, International Journal of Food Microbiology 48 (1999).

12. AP-003 (version1) Determination of MIC and MBC in microplates

13. RAO, T. P.; OKUBO, T.; CHU, D-C.; JUNEJA, L. R., Pharmacological functions of green tea polyphenols

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology, food science or related fields are intended to be within the scope of the following claims

1. A composition comprising
(a) an antimicrobial compound of the formula 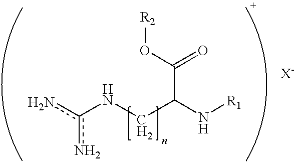
wherein R¹ is a fatty acid chain
R² is a linear or branched alkyl residue having from 1 to 12 carbon atoms
n is an integer from 0 to 10
X⁻ is selected from Br⁻, I⁻, Cl⁻ and HSO₄⁻
(b) an antimicrobial material selected from lanthionine bacteriocins, tea [Camellia sinensis] extract, hop [Humulus lupulus L.] extract, grape skin extract, grape seed extract, Uva Ursi [Arctostaphylos uva-ursi] extract and combinations thereof.

2. A composition according to claim 1 wherein the antimicrobial material is selected from lanthionine bacteriocins, tea [Camellia sinensis] extract and combinations thereof.

3-4. (canceled)

5. A composition according to claim 1 wherein R1 is —C(═O)—(CH2)p—CH3 wherein p is from 2 to 20.

6-8. (canceled)

9. A composition according to claim 1 wherein R² is a linear alkyl residue having from 1, 2 or 3 carbon atoms.

10. A composition according to claim 1 wherein R² is an ethyl residue.

11. (canceled)

12. A composition according to claim 1 wherein n is an integer from 1 to 4.

13. A composition according to claim 1 wherein n is 3.

14. A composition according to claim 1 wherein X⁻ is Cl⁻.

15. A composition according to claim 1 wherein the antimicrobial compound is

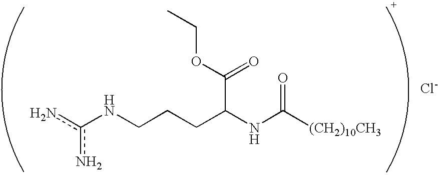

16. (canceled)

17. A composition according to claim 1 wherein the tea extract is a catechin.

18. A composition according to claim 1 wherein the tea extract is a compound selected from

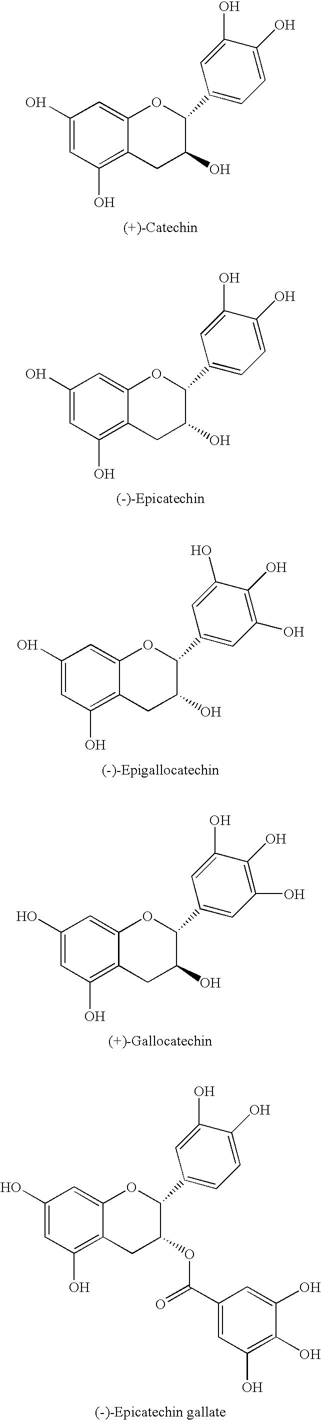

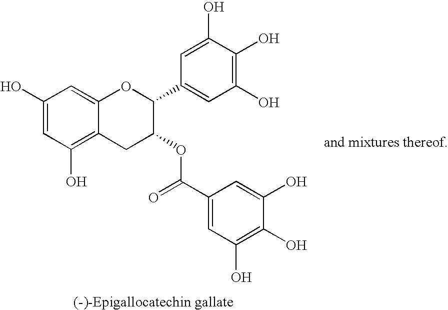

19. A composition according to claim 1 wherein the composition is an antimicrobial additive composition.

20. A composition according to claim 1 wherein the composition comprises the antimicrobial compound in an amount of at least 5% based on the composition.

21-26. (canceled)

27. A composition according to claim 1 wherein the antimicrobial material is at least nisin.

28. A composition according to claim 1 wherein the antimicrobial material is at least tea extract.

29. A composition according to claim 1 wherein the antimicrobial material is at least hop [Humulus lupulus L.] extract.

30. A composition according to claim 1 wherein the antimicrobial material is at least grape skin extract.

31. A composition according to claim 1 wherein the antimicrobial material is at least grape seed extract.

32. A composition according to claim 1 wherein the antimicrobial material is at least Uva Ursi [Arctostaphylos uva-ursi] extract.

33. A composition according to claim 1 comprising
(a) an antimicrobial compound of the formula 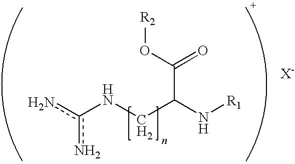
wherein R¹ is a fatty acid chain
R² is a linear or branched alkyl residue having from 1 to 12 carbon atoms
n is an integer from 0 to 10
X— is selected from Br⁻, I⁻, Cl⁻ and HSO₄⁻
(b) a lanthionine bacteriocin and
(c) a tea [Camellia sinensis] extract.

34. A composition according to claim 1 comprising 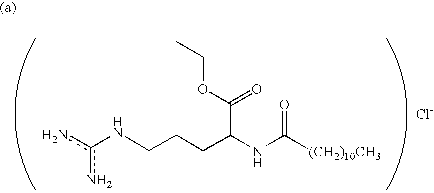
(b) nisin,

35. A composition according to claim 1 comprising 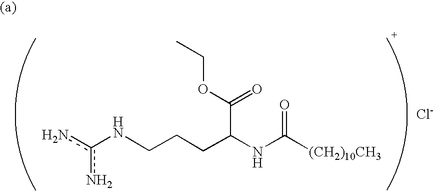
(b) a tea [Camellia sinensis] extract.

36. A composition according to claim 1 comprising 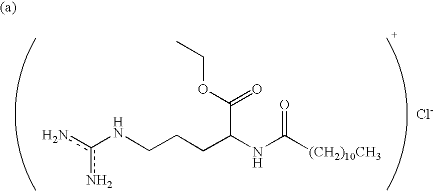
(b) a hop [Humulus lupulus L.] extract.

37. A composition according to claim 1 comprising 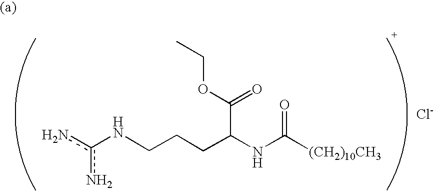
(b) a grape skin extract.

38. A composition according to claim 1 comprising 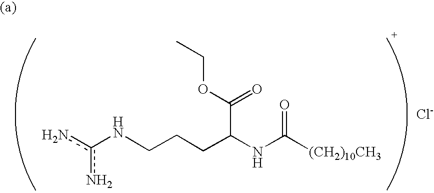
(b) a grape seed extract.

39. A composition according to claim 1 comprising 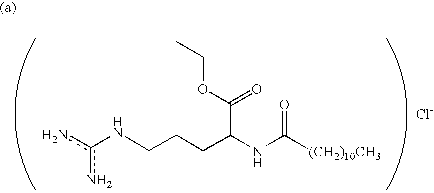
(b) a Uva Ursi [Arctostaphylos uva-ursi] extract.

40-48. (canceled)

49. A composition according to claim 1 wherein the composition further comprises an emulsifier.

50. (canceled)

51. A composition according to claim 1 wherein the composition further comprises a chelator.

52-54. (canceled)

55. A composition according to claim 1 wherein the composition further comprises a lytic enzyme.

56. (canceled)

57. A foodstuff comprising an antimicrobial additive composition according to claim 1.

58. A foodstuff according to claim 57 wherein the foodstuff is selected from raw meat, cooked meat, raw poultry products, cooked poultry products, raw seafood products, cooked seafood products, ready to eat meals, pasta sauces, pasteurised soups, mayonnaise, salad dressings, oil-in-water emulsions, margarines, low fat spreads, water-in-oil emulsions, dairy products, cheese spreads, processed cheese, dairy desserts, flavoured milks, cream, fermented milk products, cheese, butter, condensed milk products, ice cream mixes, soya products, pasteurised liquid egg, bakery products, confectionery products, fruit products, and foods with fat-based or water-containing fillings.

59. An antimicrobial protected material comprising
(i) a material to be protected from microbial growth and
(ii) an antimicrobial additive composition according to claim 1.

60. An antimicrobial protected material according to claim 59 wherein the material is selected from a paint, an adhesive, an aqueous material and water.

61. (canceled)

62. A foodstuff or antimicrobial protected material according to claim 57 wherein the composition comprises the antimicrobial compound in an amount of 100 to 200 ppm based on the composition.

63. A foodstuff or antimicrobial protected material according to claim 57 wherein the composition comprises the compound 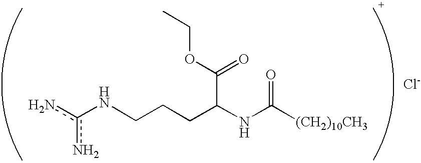
in an amount of 100 to 200 ppm based on the composition.

64. A foodstuff or antimicrobial protected material according to claim 57 wherein the composition comprises the antimicrobial material in an amount of no greater than 20000 ppm based on the composition.

65-71. (canceled)

72. A process for preventing and/or inhibiting the growth of, and/or killing a micro-organism in a material, the process comprising the step of contacting the material with
(a) an antimicrobial compound of the formula 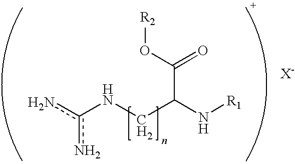
wherein R¹ is a fatty acid chain
R² is a linear or branched alkyl residue having from 1 to 12 carbon atoms
n is an integer from 0 to 10
X⁻ is selected from Br⁻, Cl⁻ and HSO₄⁻
(b) an antimicrobial material selected from lanthionine bacteriocins, tea [Camellia sinensis] extract, hop [Humulus lupulus L.] extract, grape skin extract, grape seed extract, Uva Ursi [Arctostaphylos uva-ursi] extract and combinations thereof.

73. A process according to claim 72 wherein the antimicrobial material is selected from lanthionine bacteriocins, tea [Camellia sinensis] extract and combinations thereof.

74-75. (canceled)

76. A process according to claim 72 wherein the material is a foodstuff.

77-82. (canceled)

83. A kit for preparing a composition as defined in claim 1, the kit comprising
(a) an antimicrobial compound of the formula 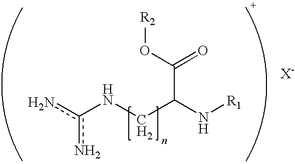
wherein R¹ is a fatty acid chain
R² is a linear or branched alkyl residue having from 1 to 12 carbon atoms
n is an integer from 0 to 10
X⁻ is selected from Br⁻, Cl⁻ and HSO₄⁻
(b) an antimicrobial material selected from lanthionine bacteriocins, tea [Camellia sinensis] extract, hop [Humulus lupulus L.] extract, grape skin extract, grape seed extract, Uva Ursi [Arctostaphylos uva-ursi] extract and combinations thereof; in separate packages or containers; optionally with instructions for admixture and/or contacting and/or use.

84. A kit according to claim 83 wherein the antimicrobial material is selected from lanthionine bacteriocins, tea [Camellia sinensis] extract and combinations thereof.

85-88. (canceled)
